(19) United States
(12) Patent Application Publication
(10) Pub. No.: US 2023/0270721 A1
(43) Pub. Date: Aug. 31, 2023

(54) INHIBITION OF BAX-MEDIATED CELL DEATH BY ELTROMBOPAG

(71) Applicant: Albert Einstein College of Medicine, Bronx, NY (US)

(72) Inventor: Evripidis Gavathiotis, Roslyn, NY (US)

(21) Appl. No.: 17/996,302

(22) PCT Filed: Apr. 15, 2021
(86) PCT No.: PCT/US2021/027453

§ 371 Date: Oct. 14, 2022

Related U.S. Application Data
(60) Provisional application No. 63/011,231, filed on Apr. 16, 2020.

(51) Int. Cl. A61K 31/4152; A61K 31/635; A61P 39/02

(52) U.S. Cl. CPC A61K 31/4152; A61K 31/635; A61P 39/02

(57) ABSTRACT

This disclosure provides methods for inhibiting BAX activity and BAX-mediated apoptosis, as well as methods for treating or preventing BAX-mediated disorders, based, in part, on an unexpected discovery that eltrombopag (EO) can work as as a potent binder to the BAX trigger site and an effective direct BAX inhibitor.

### CROSS-REFERENCE TO RELATED APPLICATIONS

The present application is a National Stage Application under 35 U.S.C. §371 of International Application No. PCT/US21/27453 filed Apr. 15, 2021, which claims priority under 35 U.S.C. §119(e) to U.S. Provisional Pat. Application No. 63/011,231, filed Apr. 16, 2020. The entire contents of these applications are incorporated herein by reference in their entireties.

### SEQUENCE LISTING

The instant application contains a Sequence Listing which has been submitted electronically in ST25 txt format and is hereby incorporated by reference in its entirety. Said ST25 txt copy, created on May 16, 2023, is named “Seq Listing 182219.00168_ST25.txt” and is 3072 bytes.

### FIELD OF THE INVENTION

This disclosure relates generally to methods for inhibiting BAX activity and BAX-mediated apoptosis, as well as methods for treating or preventing BAX-mediated disorders.

### BACKGROUND OF THE INVENTION

BAX is a BCL-2 family protein that has essential activity in mitochondrial regulation of cell death. While BAX activity ensures tissue homeostasis, when dysregulated, it contributes to aberrant cell death in several diseases, including cancer, autoimmune, neurodegenerative, and cardiovascular diseases (Singh, R., et al. Nat Rev Mol Cell Biol 20, 175-193, (2019); Delbridge, A., et al. Nat. Rev. Cancer 16, 99-109 (2016); Del Re, D. P., et al. Physiol Rev 99, 1765-1817, (2019); Hollville, E., et al. FEBS J 286, 3276-3298, (2019)). During cellular stress, BAX is transformed from an inactive cytosolic state to a mitochondrial oligomer to elicit its lethal activity. Although the BAX transformation process is not well understood, drugs that interfere with this process can be useful research tools and potential therapeutics.

Mitochondrial outer membrane permeabilization (MOMP) is a key event in mitochondrial-mediated cell death (Luna-Vargas, et al. FEBS. J. 283, 2676-2689 (2016); tait, S. W. & Green, D. R. Nat. Rev. Mol. Cell Biol. 11, 621-632 (2010)). Pro-apoptotic BCL-2 proteins BAX and BAK play a key role in this process due to their ability to transform into mitochondrial outer membrane-embedded oligomers that induce MOMP (Kale, J., et al. Cell Death Differ. 25, 65-80 (2018); Huska, J. D., et al. Methods Mol Biol 1877, 1-21, (2019)). In cells, BAX and BAK can exist as an inactive monomer, autoinhibited homodimer, or a neutralized conformation bound to anti-apoptotic BCL-2 family members such as BCL-2, BCL-xL, and MCL-1 (Edlich, F. et al. Cell 145, 104-116 (2011); Garner, T. P. et al. Mol Cell 63, 485-497 (2016); Moldoveanu, T. et al. Mol Cell 24, 677-688, (2006); Sattler, M. et al. Science 275, 983-986,983 (1997); Chen, H. C. et al. Nat Cell Biol 17, 1270-1281, (2015)). The pro-apoptotic “BH3-only” proteins such as BIM, BID, and PUMA, which comprise the third class of the BCL-2 family, utilize their BCL-2 homology 3 (BH3) domain helix to either neutralize the anti-apoptotic BCL-2 proteins and/or directly activate pro-apoptotic BAX and BAK (Ren D, et al. Science 330 1390-1393 (2010); Chen, L. et al. Mol Cell 17, 393-403, (2005)). BAX activation is a dynamic process that occurs upon binding of a BH3-only protein with its BH3 domain helix to the N-terminal BAX trigger site, inducing several conformational changes (Suzuki, M., et al. Cell 103, 645-654 (2000); Gavathiotis, E., et al. Nature 455, 1076-1081 (2008); Gavathiotis, E., et al. Mol Cell 40, 481-492 (2010); Kim, H. et al. Mol Cell 36, 487-499, (2009); Czabotar, P.E., et al. Cell 152 519-531 (2013)).

Small molecules that can modulate BAX activity can aid in elucidating the complex conformational changes of BAX in various biological mechanisms and disease models. Moreover, such small molecules can be developed into drugs to limit pathological BAX-mediated cell death.

Accordingly, there exists a strong need for methods and agents for modulating (e.g., inhibiting) BAX activity and BAX-mediated apoptosis.

### SUMMARY OF THE INVENTION

This disclosure addresses the need mentioned above in a number of aspects. In one aspect, this disclosure provides a method of treating or preventing a disorder mediated by BAX in a subject. The method comprises administering to the subject a therapeutically effective amount of eltrombopag (EO), a pharmaceutically acceptable salt thereof or pharmaceutically acceptable prodrug thereof that binds to a BAX protein and inhibits activation or function of the BAX protein.

In some embodiments, the disorder is associated with increased expression or activation of the BAX protein. In some embodiments, the disorder comprises a neuronal disorder or an autoimmune disease. In some embodiments, the neuronal disorder is selected from the group consisting of epilepsy, multiple sclerosis, Alzheimer’s disease, Huntington’s disease, Parkinson’s disease, retinal diseases, macular degeneration, spinal cord injury, Crohn’s disease, head trauma, spinocerebellar ataxias, and dentatorubral-pallidoluysian atrophy.

In some embodiments, the autoimmune disease is selected from the group consisting of multiple sclerosis, amyotrophic lateral sclerosis, retinitis pigmentosa, inflammatory bowel disease (IBD), rheumatoid arthritis, asthma, lupus, septic shock, organ transplant rejection, and AIDS.

In some embodiments, the disorder comprises ischemia (e.g., stroke, myocardial infarction, and reperfusion injury), cardiomyopathy, chemotherapy-induced cardiotoxicity, chemotherapy-induced cardiomyopathy, cardiovascular disorders, arteriosclerosis, heart failure, heart transplantation, renal hypoxia, a liver disease, a kidney disease, an intestinal disease, liver ischemia, intestinal ischemia, acute optic nerve damage, glaucoma, chemotherapy-induced ocular toxicity, hepatitis.

In some embodiments, the method further comprises administering to the subject a second therapeutic agent or therapy. In some embodiments, the second therapeutic agent is an antiinflammatory agent or an anti-tumor/anti-cancer agent. In some embodiments, the anti-tumor/anti-cancer agent is navitoclax.

In some embodiments, the second therapeutic agent is administered to the subject before, after, or concurrently with EO, a pharmaceutically acceptable salt thereof or pharmaceutically acceptable prodrug thereof.

In some embodiments, the subject is a mammal, e.g., a human. In some embodiments, the subject was previously administered an anti-cancer therapy. In some embodiments, the anti-cancer therapy comprises surgery, radiation, chemotherapy, and/or immunotherapy. In some embodiments, the chemotherapy comprises a therapeutic agent that inhibits Bcl-xL. In some embodiments, the therapeutic agent that inhibits Bcl-xL is navitoclax.

In some embodiments, EO, a pharmaceutically acceptable salt thereof or pharmaceutically acceptable prodrug thereof is administered intratumorally, intravenously, subcutaneously, intraosseously, orally, transdermally, in sustained release, in controlled release, in delayed release, as a suppository, or sublingually. In some embodiments, EO, a pharmaceutically acceptable salt thereof or pharmaceutically acceptable prodrug thereof, is administered prophylactically or therapeutically.

In another aspect, this disclosure also provides a method of treating or ameliorating a symptom of thrombocytopenia associated with treatment targeting Bcl-xL, comprising: (i) selecting a subject having a condition treatable by a therapeutic agent that inhibits Bcl-xL; and (ii) administering to the subject a therapeutically effective amount of EO, a pharmaceutically acceptable salt thereof or pharmaceutically acceptable prodrug thereof, in combination with a therapeutically effective amount of the therapeutic agent. In some embodiments, the condition is a cancer.

In some embodiments, the therapeutic agent that inhibits Bcl-xL is navitoclax. In some embodiments, the therapeutic agent is administered to the subject before, after, or concurrently with EO, a pharmaceutically acceptable salt thereof or pharmaceutically acceptable prodrug thereof. In some embodiments, the therapeutic agent or EO, a pharmaceutically acceptable salt thereof or pharmaceutically acceptable prodrug thereof, is administered in one or more doses to the subject.

In another aspect, this disclosure also provides a method of inhibiting BAX-mediated apoptosis in a cell (e.g., a neuronal cell, a cardiac cell). The method comprises administering to the cell expressing a BAX protein an effective amount of EO, a pharmaceutically acceptable salt thereof or pharmaceutically acceptable prodrug thereof that binds to the BAX protein and inhibits activation or function of the BAX protein. In some embodiments, the BAX-mediated apoptosis is caused by doxorubicin-induced cardiotoxicity.

In yet another aspect, this disclosure further provides a method of inhibiting activation or function of a BAX protein in a cell (e.g., a neuronal cell, a cardiac cell). The method comprises administering to the cell expressing a BAX protein an effective amount of EO, a pharmaceutically acceptable salt thereof or pharmaceutically acceptable prodrug thereof that binds to the BAX protein. In some embodiments, the method comprises inhibiting the activation of BAX protein that is mediated by Bim, Bid, Bmf, Puma, or Noxa.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, 1E, 1F, 1G, 1H, 1I, 1J, 1K, and 1L are a set of graphs showing eltrombopag (EO) bound and inhibited BAX. FIG. 1A shows the chemical structures of BAM7, BTSA1, and EO derived by a similarity search. The 3-methyl pyrazolone and phenylhydrazine groups are highlighted for clarity. FIG. 1B shows the results of the competitive fluorescence polarization (FP) binding assay. Data are representative of three independent experiments, each n=3 ± SEM. FIG. 1C shows the results of the microscale thermophoresis, demonstrating direct binding of EO to BAX-4C. Data are representative of three independent experiments, each n=3 ± SEM. FIGS. 1D, 1E, 1F, and 1H show the results of the BAX-mediated membrane permeabilization assay using liposomes with 50 nM BAX and 5 nM tBID (FIGS. 1D, 1E, and 1F), 50 nM BAX and 1 pM BIM-BH3 (FIG. 1G), or 250 nM BAX at 42° C. (FIG. 1H), each at 30 minutes. Data are representative of three independent experiments, each n=3 ± SEM. FIG. 1I shows the summary percentage inhibition curves for all liposomal release stimuli with IC50 included for clarity. FIGS. 1J and 1K show the results of the membrane translocation assay using NBD-labeled BAX (800 nM) activated by tBID (200 nM) (FIG. 1J) and BIM BH3 (1 µM) (FIG. 1K), each at 120 minutes. Data are representative of three independent experiments, each n=3 ± SEM. FIG. 1L shows the summary percentage inhibition curves for all BAX translocation stimuli with IC50 included for clarity. Two-sided t-test, **** P < 0.0001; ***P < 0.001 ; **P < 0.01; *P < 0.05; ns, P > 0.05.

FIGS. 2A, 2B, 2C, 2D, 2E, 2F, and 2G are a set of diagrams showing that eltrombopag bound the BAX trigger using unique contacts. FIG. 2A shows the measured chemical shift perturbations (CSPs) of ¹⁵N-labeled BAX in the presence of BAX and EO at a 1:2 molar ratio plotted as a function of BAX residue number. Residues with chemical shift perturbations over the significance threshold or 2 times the significance threshold are labeled light blue or dark blue, respectively. The black dotted line represents the average CSP. Residues associated with the N-terminal trigger site, BH3-domain, canonical site, and transmembrane domain are highlighted in yellow, green, grey, and blue, respectively. Basic residues of the N-terminal trigger site are labeled for clarity. Data are representative of three independent experiments. FIG. 2B shows the mapping of residues undergoing significant CSPs to the surface and the ribbon structure of BAX (PDB ID: 1F16). Residues with significant CSPs cluster on the N-terminal trigger site of BAX surrounding a hydrophobic pocket formed by α1 and α6. FIGS. 2C and 2D show percent inhibition of BAX-mediated membrane permeabilization using liposomes with 250 nM BAX and 5 nM tBID with various trigger site mutants. Dose response IC50 (FIG. 2C) and bar graph for 5 µM EO (FIG. 2D) are shown for clarity. Data are representative of two independent experiments, each n=3 ± SEM. FIG. 2E shows the EO binding site as determined by NMR data and docking indicated by transparent surface with ribbon representation. FIG. 2F is a closeup view of the EO binding site with residues determined by NMR data forming hydrophobic contacts with EO. EO is highlighted in cyan, and the residues forming specific interactions, R134 and R145, are highlighted in red. FIG. 2G shows BAX electrostatic surface representation of the EO binding site, where positively-charged (blue) and negatively charged (red) residues are highlighted as a gradient, and hydrophobic residues are highlighted in grey. FIG. 2H shows the results of the competitive fluorescence polarization binding assay of EO and inactive EO-methyl ester analog. Data are representative of three independent experiments n=3 ± SEM. Two-sided t-test, **** P< 0.0001; ***P < 0.001 ; **P < 0.01; *P < 0.05; ns, P> 0.05.

FIGS. 3A, 3B, and 3C are a set of diagrams showing that eltrombopag exhibits reduced chemical shift perturbations and binding to BAX R134E R145E. FIG. 3A shows measured chemical shift perturbations (CSPs) of ¹⁵N-labeled BAX R134E R145E in the presence of 1:2 BAX R134E R145E:EO plotted as a function of BAX residue number. Residues with chemical shift perturbations over the significance threshold or 2 times the significance threshold are labeled light blue or dark blue, respectively. The black dotted line represents the average CSP. Residues associated with the N-terminal trigger site, BH3-domain, canonical site, and transmembrane domain are respectively highlighted. Data is representative of three independent experiments. FIG. 3B shows mapping of significant CSPs noted in (FIG. 3A) to the surface (left) and ribbon (right) structure of BAX (PDB: 1F16). FIG. 5C shows microscale thermophoresis (MST) direct binding of EO to wild type BAX-4C and BAX-4C R134E R145E. Data are representative of two independent experiments each n=3 ± SEM. KD is shown for clarity.

FIGS. 4A, 4B, 4C, 4D, and 4E are a set of diagrams showing that eltrombopag methyl ester analog (EO-methyl ester) exhibits reduced chemical shift perturbations and binding to BAX. FIG. 4A shows chemical structures of of EO and EO-methy-ester. FIG. 4B shows measured chemical shift perturbations (CSPs) of ¹⁵N-labeled BAX in the presence of 1:2 BAX:EO-methyl ester plotted as a function of BAX residue number. Residues with chemical shift perturbations over the significance threshold or 2 times the significance threshold are labeled light blue or dark blue, respectively. The black dotted line represents the average CSP. Residues associated with the N-terminal trigger site, BH3-domain, canonical site, and transmembrane domain are highlighted in yellow, green, grey, and blue, respectively. Data is representative of three independent experiments. FIG. 4C shows mapping of significant CSPs noted in (FIG. 4B) to the surface (left) and ribbon (right) structure of BAX (PDB: 1F16). FIG. 4D shows representative kinetic traces of ANTS/DPX liposomal release with respect to time in the presence of 10 µM EO or EO-methyl ester. Conditions are 50 nM BAX and 5 nM tBID. Data are representative of two independent experiments each n=3 ± SEM. e, Percentage inhibition of tBID induced BAX mediated liposomal permeabilization by EO and EO-methyl ester. Data are representative of two independent experiments each n=3 ± SEM. Two-sided t-test, **** P < 0.0001; ***P < 0.001;**P < 0.01; *P < 0.05; ns, P > 0.05.

FIGS. 5A, 5B, 5C, 5D, 5E, 5F, 5G, and 5H are a set of diagrams showing that eltrombopag stabilized BAX in an inactive conformation. FIG. 5A shows an overlay of structures of the BAX-EO complex from 10 nsec intervals from 0-100 nsec molecular dynamics (MD) simulation. EO color spectrum corresponding to time is shown. BAX ribbon structures are colored grey with residues of interest represented as sticks for clarity. FIGS. 5B and 5C show distance relative to time of EO carboxylate-R145 (carbonyl carbon-4-carbon) (FIG. 5B) and EO pyrazolone carbonyl-R134 (carbonyl oxygen-4-carbon) (FIG. 5C). Blue shades represent individual MD simulation distances, and black represents mean of n=3 simulations. FIGS. 5D and 5E show the histogram representation of α-carbon distance frequency during MD simulation between R134 and negatively charged residues D48 (FIG. 5D) and E44 (FIG. 5E) on α1-α2 loop. Data represent mean of n=3 for both BAX and BAX-EO complex. FIG. 5F shows percentage change in root mean square fluctuation (RMSF) of BAX-EO versus BAX, plotted with respect to BAX residue number. Color gradient is representative of the change in RMSF, with blue and red corresponding to decrease and increase in RMSF, respectively. Data represent mean of n=3 simulations for both BAX and BAX-EO. The average percentage change is represented by the dotted black line, with ±SD represented as the red and blue dashed lines. Residues associated with the N-terminal trigger site, BH3-domain, canonical site, and transmembrane domain are highlighted in yellow, green, grey, and blue, respectively. FIG. 5G shows the changes in structure and dynamics of α7/α4-α5 loop interface: representative α-carbon distance frequency histogram for F105-Q155 (left), transparent surface with ribbon representation of α7/α4-α5 loop interface with residues of note highlighted in blue (center), and graphical representation of distances between residues at α7/α4-α5 loop interface (right). FIG. 5H shows the changes in structure and dynamics of the canonical site opening formed by α3, loop 3, α4, and α9: representative α-carbon distance frequency histogram for T85-K189 (left), transparent surface with ribbon representation of canonical site opening with residues of note highlighted in blue (center), and graphical representation of distances between residues at the canonical site opening (right). Distances represent the mean difference of n=3 BAX and BAX-EO MD simulations (FIGS. 5G and 5H). Distances are plotted with respect to time, and distance frequency histograms for all distances are available in supplemental figures as referenced.

FIGS. 6A, 6B, 6C, 6D, 6E, and 6F are a set of graphs showing that eltrombopag inhibited BAX-mediated cell death. FIGS. 6A and 6B show percentage inhibition of cytochrome c release, as determined by ELISA in the presence of BIM-BH3 peptide and increasing doses of EO in BAKKO mouse embryonic fibroblasts (MEFs) (FIG. 6A). The dose response EC50 curves for percentage inhibition of BIM-BH3 induced cytochrome c release by EO with EC50 are shown for clarity (FIG. 6B). Data represent mean of n=3 ± SEM and are representative of three independent experiments. FIG. 6C shows the results of the viability assay of 3T3 cells upon treatment with 1 µM ABT-263 (or navitoclax) and 1 µM S63845 in the presence or absence of various doses of EO for 24 hr. Viability upon ABT-263 and S63845 combination in the absence of EO is indicated by the red dashed line. Data represent mean of n=3 ± SEM and are representative of three independent experiments. FIG. 6D shows the results of the caspase 3/7 assay of 3T3 cells upon treatment with 1 µM ABT-263 and 1 µM S63845 in the presence or absence of various doses of EO for 4 hr. Data represent mean of n=3 ± SEM and are representative of three independent experiments. Two-sided t-test, **** P < 0.0001; ***P < 0.001; **P < 0.01; *P < 0.05; ns, P > 0.05. FIG. 6E shows platelet counts of C57BL/6J mice treated with vehicle, ABT-263 (25 mg/kg, single dose), EO (100 mg/kg, then 3 hrs later 50 mg/kg), or a combination of ABT-263 (or navitoclax) and EO by oral gavage. Data represent mean of n= 5 ± SEM. FIG. 6F shows % platelet protection plotted based on the data in FIG. 6E.

FIGS. 7A, 7B, 7C, 7D, 7E, and 7F are a set of diagrams showing that eltrombopag inhibits BAX translocation and BAX-mediated apoptosis in cells. FIG. 7A shows the confocal micrographs of BAK KO MEFs treated with DMSO, 2 µM STS for 4.5 h without or with 10 µM EO 6.5 h, respectively. BAX translocation is based on antibody-based detection of BAX and mitochondrial protein TOMM20. Representative confocal micrographs from three independent biological experiments. Scale bar, 20 µm. FIG. 7B shows quantification of BAX translocation (% of cells with BAX foci colocalizing with TOMM20 foci) in BAK KO MEFs induced by 2 µM staurosporine (STS) and inhibited by 10 µM EO. Data represent ±SEM of three independent biological replicates. Two-sided t test, ****P < 0.0001; ***P < 0.001; **P < 0.01; *P < 0.05; ns, P > 0.05. FIG. 7C shows representative immunoblot analysis of BAX translocation in BAK KO MEF cells in response to BIM BH3 titration in the presence of 10 or 20 µM EO. Actin and VDAC are loading controls for cytosolic and mitochondrial fractions, respectively. FIGS. 7D and 7E show a caspase 3/7 assay of BAK-/- mouse embryonic fibroblasts (MEFs) (FIG. 7D) and BAX-/-MEFs (FIG. 7E) in response to 3 µM STS and the presence or absence of various doses of EO for 6 hr. Data represent mean of n=3 ± SEM and are representative of three independent experiments. Two-sided t-test, **** P < 0.0001; ***P< 0.001;**P < 0.01; *P< 0.05; ns, P> 0.05. FIG. 7F shows apoptosis detection using RealTime Glo (Promega) assay in human cardiomyocytes cells (IPSC-CM) in response to 5 µM cardiotoxic Doxorubicin and the presence or absence of 10 µM EO at various time points. Data represent mean of n=3 ± SEM.

### DETAILED DESCRIPTION OF THE INVENTION

This disclosure provides methods for inhibiting BAX activity and BAX-mediated apoptosis, as well as methods for treating or preventing BAX-mediated disorders, based, in part, on an unexpected discovery that eltrombopag (EO), an FDA-approved drug, can work as as a potent binder to the BAX trigger site and an effective direct BAX inhibitor.

As demonstrated in this disclosure, EO inhibited BAX activation by a novel two-fold mechanism. BAX inhibition by EO was dependent on the concentration of EO, BAX, and BH3-activators, and EO directly engaged the BAX trigger site binding, consistent with a direct competitive mechanism. Furthermore, EO inhibited heat-induced translocation and activation of BAX, promoted stabilization of the α1-α2 loop in closed conformation and interaction with α6, and induced conformational changes associated with reduced BAX activity, such as those observed at the α7/α4-α5 loop and canonical site-α9 interfaces. Thus, this disclosure presents a unique mechanism of BAX inhibition by EO that directly competes with BH3-only proteins for binding to BAX and simultaneously promotes allosteric conformational changes that stabilize the inactive soluble BAX structure. EO engages the trigger site with a unique binding mode distinct from BAX activators, using hydrophobic interactions with a shallow hydrophobic groove formed by residues of α6, α1, and the closed al -α2 loop. Thus, this disclosure also offers a blueprint for rational design of a novel class of BAX inhibitors. This disclosure further demonstrated that EO can inhibit BAX-mediated apoptosis and prevent platelet death in vivo.

### A. Methods for Treating or Preventing Bax-Mediated Disorders and Methods for Inhibiting Bax Activity and Bax-Mediated Apoptosis

In one aspect, this disclosure provides a method of treating or preventing a disorder mediated by BAX in a subject. The method comprises administering to the subject a therapeutically effective amount of EO, a pharmaceutically acceptable salt thereof, a pharmaceutically active derivative thereof or pharmaceutically acceptable prodrug thereof (e.g., methyl ester EO) that binds to a BAX protein and inhibits activation or function of the BAX protein.

EO is an FDA-approved thrombopoietin receptor agonist and iron chelator. It is used to treat low blood platelet counts in adults with chronic immune (idiopathic) thrombocytopenia (ITP), when certain other medicines, or surgery to remove the spleen, have not worked well enough. It has excellent oral bioavailability with a peak concentration occurring 2-6 h after oral administration and a half-life of 21-32 h (Cheng et al. Ther Adv Hematol. 2012 Jun; 3(3): 155-164.) The chemical structure of EO is represented by formula I:

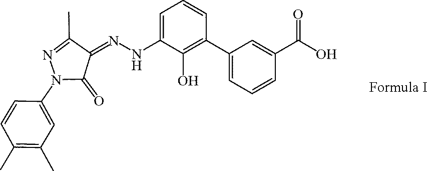 [SMILES: CC1=NN(c2ccc(C)c(C)c2)C(=O)/C1=N\Nc1cccc(-c2cccc(C(=O)O)c2)c1O]

“Pharmaceutically acceptable salts” or “pharmaceutically acceptable complexes” refers to salts or complexes of EO that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, trifluoroacetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and polygalacturonic acid. Said compounds can also be administered as pharmaceutically acceptable quaternary salts known by a person skilled in the art, which specifically include the quaternary ammonium salt of the formula - NR,R′,R″ ⁺ Z″, wherein R, R′, R″ is independently hydrogen, alkyl, or benzyl, and Z is a counterion, including chloride, bromide, iodide, -O-alkyl, toluenesulfonate, methylsulfonate, sulfonate, phosphate, or carboxylate (e.g., benzoate, succinate, acetate, glycolate, maleate, malate, fumarate, citrate, tartrate, ascorbate, cinnamate, mandelate, and diphenylacetate).

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, is capable of providing directly or indirectly the activity disclosed herein.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

As used herein, “treatment” or “treating,” or “palliating” or “ameliorating” are used interchangeably. These terms refer to an approach for obtaining beneficial or desired results, including, but not limited to, a therapeutic benefit and/or a prophylactic benefit. By therapeutic benefit is meant any therapeutically relevant improvement in or effect on one or more diseases (e.g., inflammatory diseases, neurodegenerative diseases, cardiovascular diseases), conditions, or symptoms under treatment. For prophylactic benefit, the agent or the compositions thereof may be administered to a subject at risk of developing a particular disease, condition, or symptom, or to a subject reporting one or more of the physiological symptoms of a disease, even though the disease, condition, or symptom may not have yet been manifested.

The terms “inhibit,” “decrease,” “reduced,” “reduction,” or “decrease” are all used herein generally to mean a decrease by a statistically significant amount. However, for avoidance of doubt, “reduced,” “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “activate,” “increased,” “increase” or “enhance” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” is meant to refer to any change in biological state, i.e., increasing, decreasing, and the like.

Through the modulation of the BAX function, disorders mediated by BAX can be treated or prevented. Such disorders may include neuronal disorders and/or disorders of the immune system. The modulation may involve the inhibition of the activity (activation) and/or of the expression of BAX. For example, the modulation of the BAX function or activity may include the inhibition or disruption of the interaction of Bim, Bid, Bmf, Puma, or Noxa with BAX, which has been shown to play a role within the context of the BAX activation leading to cytochrome c release (J.C. Martinou et al. The Journal of Cell Biology, 144(5), 891-901 (1999)). As a result of the inhibition of the BAX activation by Bim, Bid, Bmf, Puma, or Noxa upon using EO, the cytochrome c release could be inhibited or essentially blocked, thus providing a means to modulate the apoptosis pathways. As a result, by modulation of the apoptosis pathways, a wide variety of disorders associated with abnormal apoptosis can be treated.

In some embodiments, EO is suitable for use in treating disorders associated with an abnormal BAX function or abnormal (e.g., elevated) BAX activation, an abnormal expression or activity of BAX. Thus, the treatment or prevention of disorders involves modulation (e.g., inhibition, disruption) of the BAX function or activation, in particular with the abnormal expression or activity of BAX, using EO, a pharmaceutically acceptable salt thereof, a pharmaceutically active derivative thereof or pharmaceutically acceptable prodrug thereof.

For example, EO can be used for treating the disorders, such as neuronal disorders, autoimmune diseases, and cardiovascular diseases. In some embodiments, the neuronal disorder includes epilepsy, multiple sclerosis, Alzheimer’s disease, Huntington’s disease, Parkinson’s disease, retinal diseases, macular degeneration, spinal cord injury, Crohn’s disease, head trauma, spinocerebellar ataxias, and dentatorubral-pallidoluysian atrophy.

In some embodiments, the autoimmune disease includes multiple sclerosis, amyotrophic lateral sclerosis, retinitis pigmentosa, inflammatory bowel disease (IBD), rheumatoid arthritis, asthma, lupus, septic shock, organ transplant rejection, and AIDS.

In some embodiments, the disorder includes ischemia (e.g., stroke, myocardial infarction, and reperfusion injury), cardiomyopathy, chemotherapy-induced cardiotoxicity, chemotherapy-induced cardiomyopathy, cardiovascular disorders, arteriosclerosis, heart failure, heart transplantation, renal hypoxia, a liver disease, a kidney disease, an intestinal disease, liver ischemia, intestinal ischemia, acute optic nerve damage, glaucoma, chemotherapy-induced ocular toxicity, hepatitis.

The term “disease” as used herein is intended to be generally synonymous and is used interchangeably with the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., neuronal disorders, autoimmune diseases, and cardiovascular diseases), and/or who has been diagnosed with inflammatory disorders. In some embodiments, the subject is a mammal. In some embodiments, the subject is human.

In some embodiments, the method further comprises administering to the subject a second therapeutic agent or therapy. In some embodiments, the second therapeutic agent is an anti-inflammatory agent or an anti-tumor/anti-cancer agent. In some embodiments, the anti-tumor/anti-cancer agent is navitoclax.

In some embodiments, the second therapeutic agent is administered to the subject before, after, or concurrently with EO, a pharmaceutically acceptable salt thereof, a pharmaceutically active derivative thereof or pharmaceutically acceptable prodrug thereof.

In some embodiments, the subject was previously administered an anti-cancer therapy. In some embodiments, the anti-cancer therapy comprises surgery, radiation, chemotherapy, and/or immunotherapy. In some embodiments, the chemotherapy comprises a therapeutic agent that inhibits Bcl-xL. In some embodiments, the therapeutic agent that inhibits Bcl-xL is navitoclax ( or ABT-263).

Navitoclax is an orally active, small synthetic molecule and an antagonist of a subset of the B-cell leukemia 2 (Bcl-2) family of proteins (e.g., Bcl-xL) with potential antineoplastic activity. Navitoclax selectively binds to apoptosis suppressor proteins Bcl-2, Bcl-xL, and Bcl-w, which are frequently overexpressed in a wide variety of cancers, including those of the lymph, breast, lung, prostate, and colon, and are linked to tumor drug resistance. Inhibition of these apoptosis suppressors prevents their binding to the apoptotic effectors BAX and BAK proteins, thereby triggering apoptotic processes in cells overexpressing Bcl-2, Bcl-xL, and Bcl-w. This eventually reduces tumor cell proliferation. Navitoclax has been used in trials studying the treatment of solid tumors, Non-Hodgkin’s lymphoma, EGFR activating mutation, chronic lymphoid leukemia, and hematological malignancies, and other cancers. The chemical structure of navitoclax is represented by formula II:

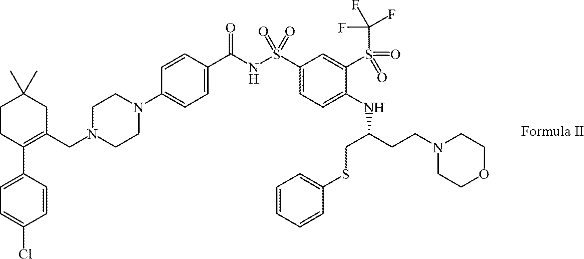 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, EO, a pharmaceutically acceptable salt thereof, a pharmaceutically active derivative thereof or pharmaceutically acceptable prodrug thereof is administered intratumorally, intravenously, subcutaneously, intraosseously, orally, transdermally, in sustained release, in controlled release, in delayed release, as a suppository, or sublingually. In some embodiments, EO, a pharmaceutically acceptable salt thereof, a pharmaceutically active derivative thereof or pharmaceutically acceptable prodrug thereof, is administered prophylactically or therapeutically.

In another aspect, this disclosure also provides a method of treating or ameliorating a symptom (e.g., platelet loss) of thrombocytopenia associated with treatment targeting Bcl-xL, comprising: (i) selecting a subject having a condition treatable by a therapeutic agent that inhibits Bcl-xL; and (ii) administering to the subject a therapeutically effective amount of EO, a pharmaceutically acceptable salt thereof, a pharmaceutically active derivative thereof or pharmaceutically acceptable prodrug thereof, in combination with a therapeutically effective amount of the therapeutic agent.

In some embodiments, the condition is a cancer (e.g., solid tumors, Non-Hodgkin’s lymphoma, EGFR activating mutation, chronic lymphoid leukemia, and hematological malignancies). In some embodiments, cancers are characterized by overexpression of a Bcl-2 family protein (e.g., Bcl-xL), such as lymphoma, skin cancer, pancreatic cancer, colon cancer, melanoma, liver cancer, bladder cancer, non-small cell lung cancer, myeloma, leukemia, and head and neck cancer.

In some embodiments, the therapeutic agent that inhibits Bcl-xL is navitoclax. In some embodiments, the therapeutic agent is administered to the subject before, after, or concurrently with EO, a pharmaceutically acceptable salt thereof, a pharmaceutically active derivative thereof or pharmaceutically acceptable prodrug thereof. In some embodiments, the therapeutic agent or EO, a pharmaceutically acceptable salt thereof, a pharmaceutically active derivative thereof or pharmaceutically acceptable prodrug thereof, is administered in one or more doses to the subject.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In another aspect, this disclosure also provides a method of inhibiting BAX-mediated apoptosis in a cell (e.g., a neuronal cell, a cardiac cell). The method comprises administering to the cell expressing a BAX protein an effective amount of EO, a pharmaceutically acceptable salt thereof, a pharmaceutically active derivative thereof or pharmaceutically acceptable prodrug thereof that binds to the BAX protein and inhibits activation or function of the BAX protein. In some embodiments, the BAX-mediated apoptosis is caused by doxorubicin-induced cardiotoxicity.

“Apoptosis” refers to the process by which cells are programmed to die or lose viability. Commonly triggered by cytochrome leakage from the mitochondria and accompanied by signaling cascades (caspases and other proteins) resulting in decreased mitochondrial and energy potential via the electron transport system, a build-up of reactive oxygen species and free radical and loss of membrane integrity.

In yet another aspect, this disclosure further provides a method of inhibiting activation or function of a BAX protein in a cell. The method comprises administering to the cell (e.g., a neuronal cell, a cardiac cell) expressing a BAX protein an effective amount of EO, a pharmaceutically acceptable salt thereof, a pharmaceutically active derivative thereof or pharmaceutically acceptable prodrug thereof that binds to the BAX protein.

In some embodiments, the method comprises inhibiting the activation of BAX protein that is mediated by Bim, Bid, Bmf, Puma, or Noxa.

### B. Compositions and Administration Regimens

Also within the scope of this disclosure are the pharmaceutical compositions for use in the methods, as described above. The pharmaceutical compositions may include EO, or an analog, a derivative, or a pharmaceutically acceptable prodrug, a pharmaceutically active metabolite, a pharmaceutically acceptable salt thereof.

Pharmaceutical compositions for use in accordance with the present methods may be formulated in a conventional manner using one or more physiologically acceptable carriers or excipients. Thus, EO and its analogs/ derivatives that modulate the activation and/or function of BAX, and their physiologically acceptable salts and solvates may be formulated for administration by, for example, injection, inhalation or insufflation (either through the mouth or the nose) or oral, buccal, parenteral or rectal administration. In one embodiment, the agent is administered locally, e.g., at the site where the target cells are present, such as by the use of a patch.

Pharmaceutical compositions can be formulated for a variety of loads of administration, including systemic and topical or localized administration. Techniques and formulations generally may be found in Remmington’s Pharmaceutical Sciences, Meade Publishing Co., Easton, PA. For systemic administration, injection is preferred, including intramuscular, intravenous, intraperitoneal, and subcutaneous. For injection, the agents can be formulated in liquid solutions, preferably in physiologically compatible buffers such as Hank’s solution or Ringer’s solution. In addition, the agents may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Pharmaceutical compositions that may oxidize and lose biological activity, especially in a liquid or semisolid form, may be prepared in a nitrogen atmosphere or sealed in a type of capsule and/or foil package that excludes oxygen (e.g., Capsugel™).

For administration by inhalation, the agents may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the agent and a suitable powder base such as lactose or starch.

Pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The agents may also be formulated in rectal compositions such as suppositories or retention enemas, e.g., containing conventional suppository bases such as cocoa butter or other glycerides.

In addition to the formulations described previously, pharmaceutical compositions may also be formulated as a depot preparation. Such long-acting formulations may be administered by implantation (for example, subcutaneously or intramuscularly) or by intramuscular injection. Thus, for example, the agents may be formulated with suitable polymeric or hydrophobic materials (for example, as an emulsion in an acceptable oil) or ion exchange resins, or as sparingly soluble derivatives, for example, as a sparingly soluble salt. Controlled release formula also includes patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions (including cosmetic preparations) may comprise from about 0.00001 to 100%, such as from 0.001 to 10% or from 0.1% to 5%, by weight of one or more agents described herein.

A pharmaceutical composition described herein can also be incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols, and other nontoxic organic solvents, glycerin, mineral oil, silicone, petroleum jelly, lanolin, fatty acids, vegetable oils, parabens, waxes, and the like.

Pharmaceutical compositions may be incorporated into gel formulations, which generally are semisolid systems consisting of either suspension made up of small inorganic particles (two-phase systems) or large organic molecules distributed substantially uniformly throughout a carrier liquid (single-phase gels). Single-phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose, and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives. An optimum topical formulation comprises approximately: 2 wt.% to 60 wt.%, preferably 2 wt.% to 50 wt.%, solubilizer and/or skin permeation enhancer; 2 wt.% to 50 wt.%, preferably 2 wt.% to 20 wt.%, emulsifiers; 2 wt.% to 20 wt.% emollient; and 0.01 to 0.2 wt. % preservative, with the active agent and carrier (e.g., water) making of the remainder of the formulation. A skin permeation enhancer serves to facilitate passage of therapeutic levels of active agent to pass through a reasonably sized area of unbroken skin. Suitable enhancers are well known in the art and include, for example, lower alkanols such as methanol ethanol and 2-propanol; alkyl methyl sulfoxides such as dimethylsulfoxide (DMSO), decylmethylsulfoxide (C.sub.lO MSO) and tetradecylmethyl sulfoxide; pyrrolidones such as 2-pyrrolidone, N-methyl-2-pyrrolidone and N-(-hydroxyethyl)pyrrolidone; urea; N,N- diethyl-m-toluamide; C.sub.2 -C. sub.6 alkane diols; miscellaneous solvents such as dimethylformamide (DMF), N,N-dimethylacetamide (DMA) and tetrahydrofurfuryl alcohol; and the 1 -substituted azacycloheptan-2-ones, particularly 1-n-dodecylcyclazacycloheptan-2-one (laurocapram; available under the trademark AzoneRTM from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2- pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol- 2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate). In certain topical formulations, the active agent is present in an amount in the range of approximately 0.25 wt.% to 75 wt.% of the formulation, preferably in the range of approximately 0.25 wt.% to 30 wt.% of the formulation, more preferably in the range of approximately 0.5 wt.% to 15 wt.% of the formulation, and most preferably in the range of approximately 1.0 wt.% to 10 wt.% of the formulation. Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

In some embodiments, a pharmaceutical formulation is provided for oral or parenteral administration, in which case the formulation may comprise an activating compound-containing microemulsion as described above, and may contain alternative pharmaceutically acceptable carriers, vehicles, additives, etc. particularly suited to oral or parenteral drug administration. Alternatively, an activating compound-containing microemulsion may be administered orally or parenterally substantially, as described above, without modification.

Dosages for a particular individual can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to an individual is sufficient to effect a beneficial therapeutic response in the individual over time, or, e.g., to reduce symptoms, or other appropriate activity, depending on the application. The dose is determined by the efficacy of the particular formulation, and the activity, stability or serum half-life of the miRNA employed and the condition of the individual, as well as the body weight or surface area of the individual to be treated. The size of the dose is also determined by the existence, nature, and extent of any adverse side-effects that accompany the administration of a particular vector, formulation, or the like in a particular individual.

### C. Definitions

To aid in understanding the detailed description of the compositions and methods according to the disclosure, a few express definitions are provided to facilitate an unambiguous disclosure of the various aspects of the disclosure. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease or condition being treated, the weight and age of the subject, the severity of the disease or condition, the manner of administration, and the like, which can readily be determined by one of ordinary skill in the art. For example, certain compositions described herein may be administered in a sufficient amount to produce a desired effect at a reasonable benefit/risk ratio applicable to such treatment.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product(s).” If the polynucleotide is derived from genomic DNA, expression may include splicing of the mRNA in a eukaryotic cell.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the invention, and are physiologically acceptable to the subject. Supplementary active compounds may also be incorporated into the compositions.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. Examples

### Example 1

This example describes the materials and methods used in EXAMPLES 2-6 below.

### Reagents

Hydrocarbon-stapled peptides corresponding to the BH3 domain of BIM, BIM SAHB: FITC-Ahx-EIWIAQELRS5IGDS5FNAYYA-CONH (SEQ ID NO: 1), where S5 represents the non-natural amino acid inserted for olefin metathesis, were synthesized, purified at > 95% purity by CPC Scientific Inc. and characterized as previously described (Gavathiotis, E., et al. Nat Chem Biol 8, 639-645 (2012)). Peptides corresponding to the BH3-domain of BIM, BIM-BH3, Ac- RPEIWIAQELRRIGDEFNAYYARR (SEQ ID NO: 2) were synthesized by GenScript at > 95% purity. Recombinant tBID in > 95% purity, as determined by SDS-PAGE under reducing conditions, was purchased by R&D Systems (cat. # 882-B8-050). Eltrombopag (cat. # 100941) and eltrombopag methyl ester (cat. # SC498745) were purchased from Medkoo Biosciences and Santa Cruz Biotechnology, respectively, and their molecular identity and purity > 95% was confirmed by NMR. ABT-263 (cat. # S1001) and S63845 (cat. # A8737) were purchased from Selleckchem and APExBIO, respectively. Compounds were stored as powdered, reconstituted into 100% DMSO, and diluted as described.

### Production of Recombinant BAX

Human full-length (1-192) wild-type BAX (Q07812; SEQ ID NO: 3) was cloned in pTYB1 vector (New England BioLabs) between the NdeI and SapI restriction sites. Mutations were generated using the QuickChange Lightning site-directed mutagenesis kit (Agilent). Recombinant proteins were expressed in BL21 (DE3) CodonPlus (DE3)-RIPL, grown in Luria Broth (LB) media and induced with 1 mM isopropyl p-d-1-thiogalactopyranoside (IPTG). The bacterial pellet was resuspended in lysis buffer (20 mM TrisCI pH 7.6, 250 mM NaCI, 1 mM EDTA, and Roche complete EDTA free protease inhibitor cocktail), lysed by high-pressure homogenization, and clarified by ultracentrifugation at 45,000 × g for 45 min. The supernatant was applied to 5 ml of pre-equilibrated chitin beads (New England BioLabs) in a gravity-flow column and washed with 3 column volumes of lysis buffer. BAX was cleaved by overnight incubation using 50 mM DTT in lysis buffer. Cleaved BAX was eluted with lysis buffer, concentrated with a Centricon spin concentrator (Millipore), and purified by gel filtration using a Superdex 75 10/300 GL, column (GE Healthcare Life Sciences), pre-equilibrated with gel filtration buffer (20 mM HEPES, 150 mM KCI, pH 7.2) at 4° C. Fractions containing BAX monomer are pooled and concentrated using a 10-KDa cut-off Centricon spin concentrator (Millipore) for prompt use in biochemical and structural studies.

### Fluorescence Polarization Binding Assays

Fluorescence polarization assays (FPA) were performed as previously described (Gavathiotis, E., et al. Nat Chem Biol 8, 639-645 (2012)). Direct binding isotherms of BIM-SAHB were measured by incubated FITC-BIM-SAHB (25 nM) with serial dilutions of full-length BAX alone or in the presence of 0.5 or 1 µM EO. Competition binding assays were performed by titrating ED into BAX (150 nM) and FITC-BIM-SAHB (25 nM). Measurements were taken at 10-minute intervals over 60 minutes on a TECAN F200 PRO microplate reader. Reported curves represent 10-minute time point. K_{D} values and IC50 were determined using GraphPad Prism nonlinear fit four-parameter agonist or antagonist versus response with restraints for 100% and 0% bound calculated by the mP of saturated BAX + FITC-BIM-SAHB and FITC-BIM-SAHB alone.

### Microscale Thermophoresis

Recombinant BAX C62S-C126S-S5C (4C), previously established for evaluating BAX (Garner, T.P., et al. Nat Chem Biol 15, 322-330 (2019)) binding compounds with MST, or BAX C62S-C126S-S5C-R134E-R145E (R134E-R145E-4C) was labeled at cysteine using the Monolith Protein Labeling Kit Red Maleimide (NanoTemper Technologies) according to the instructions of the manufacturer (Garner, T.P., et al. Nat Chem Biol 15, 322-330 (2019)). Briefly, 10 µM protein was incubated with 0.9 equivalents of dye in MST buffer (100 mM potassium phosphate, pH 7.4, 150 mM NaCI) in the dark at room temperature (22-25° C.) for 1 hr. Unreacted dye was quenched using 5 mM DTT and removed using the manufacturer-provided buffer exchange column. To determine the K_{D} of BAX to EO, 50 nM labeled BAX was incubated with increasing concentrations of EO in MST buffer supplemented with 0.25% CHAPS. Samples were loaded into standard glass capillaries (Monolith NT.155 Capillaries) and analyzed by MST using a Monolith NT.115 Blue/Red, LED power and IR laser power of 80%. Fraction bound and error was generated by NanoTemper software (MO.Affinity Analysis), and K_{D} values were determined using GraphPad Prism nonlinear fit four-parameter agonist versus response with restraints for 0 and 1 fraction bound.

### Liposomal Permeabilization Assay

Lipids (Avanti Polar Lipids) at the following ratio, phosphatidylcholine 48%, phosphatidylinositol 10%, dioleoyl phosphatidylserine 10%, phosphatidylethanolamine, 28%, and tetraoleoyl cardiolipin 4%, were mixed in a total of 1 mg, dried and resuspended in 10 mM HEPES, pH 7, 200 mM KCI, and 5 mM MgCl₂ with 12.5 mM 8-aminonaphthalene-1,3,6-trisulfonic acid (ANTS) dye and 45 mM p-xylene-bis-pyridinium bromide (DPX) quencher (Molecular Probes) using a water bath sonicator. Liposomes were formed by extrusion of the suspension using Avanti Mini-Extruder (Cat # 610000) with polycarbonate membranes of 0.1 pm pore size (Avanti Polar Lipids). ANTS/DPX encapsulated liposomes were purified from non-encapsulated ANTS/DPX by gel filtration of a 10 mL CL2B-Sepharose (GE Healthcare Life Sciences) gravity-flow column. BAX (50-250 nM) was combined with tBID, BIM, and EO at the indicated concentrations to a volume of 90 µL. Reactions were initiated by the addition of 10 pL of the encapsulated ANTS/DPX liposome stock. ANTS/DPX release was quantified based on the increase in fluorescence intensity that occurs when the ANTS fluorophore is separated from the DPX quencher upon release from the liposomes into solution. Fluorescence (λex = 355 nm and λem = 520 nm) was measured at 1 minute intervals at room temperature (22-25° C.) indicated using a Tecan Infinite M1000 plate reader. In the case of heat activation, reactions were set up as described in the absence of tBID or BIM, and experiments were recorded at 42° C. The percentage release of ANTS/DPX at any given time point was calculated as percentage release=((F-F₀)/(F₁₀₀-F₀))(100), where F₀ and F₁₀₀ are baseline and maximal fluorescence, respectively. Triton X-100 (1%) was used to determine the maximum amount of liposomal release per assay and was set to 100%.

### Liposomal Translocation Assay

Lipids (Avanti Polar Lipids) at the following ratio, phosphatidylcholine 48%, phosphatidylinositol 10%, dioleoyl phosphatidylserine 10%, phosphatidylethanolamine, 28%, and tetraoleoyl cardiolipin 4%, were mixed in a total of 1 mg, dried and resuspended in 10 mM HEPES, pH 7, 200 mM KCl, and 5 mM MgCl₂. The resulting slurry was vortexed for 10 minutes and sonicated in a sonicating water bath for 10 minutes. Liposomes were formed by extrusion of the suspension using Avanti Mini-Extruder with polycarbonate membranes of 0.1 µm pore size (Avanti Polar Lipids) followed by passage through a CL2B Sepharose column (GE Healthcare). Recombinant wild type BAX was labeled at cysteine by overnight incubation at 4° C. with 10 equivalents of iodoamino-NBD (IANBD, Thermo Fisher) and 3 equivalents of TCEP to maintain reduced cysteine. Labeled BAX (BAX-NBD) was separated from unreacted IANBD by gel filtration (Econo-Pac 10 DG desalting column, BioRad) and used immediately. Translocation reactions were performed by combining 800 nM BAX with 1 µM BIM or 200 nM tBID in the presence and absence of varying doses of EO. Reactions were initiated by the addition of 10 µL of the liposome stock. The NBD fluorophore exhibits low fluorescence in solution due to quenching by water. Upon BAX-NBD translocation, the NBD fluorophore was excluded from bulk water through contact with the liposomal membrane leading to an increase in fluorescence intensity. Fluorescence (λex = 475 nm and λem = 530 nm) was measured at 1-minute intervals at 37° C. indicated using a Tecan Infinite M1000 plate reader. The percentage translocation at any given time point was calculated as percentage translocation=[((F-F₀)/(F₁₀₀-F₀))(100)]-[((F_{S}-F_{S0})/(F_{S100}-F_{S0}))(100)] where F₀ and F₁₀₀ are baseline and maximal fluorescence, respectively and F_{S}, F_{S0}, and F_{S100} are the current fluorescence, baseline fluorescence, maximal fluorescence of solution BAX incubated in the absence of liposomes. The subtraction of the percent translocation of solution BAX is required to correct for NBD fluorescence bleaching that occurs throughout the reaction. Triton X-100 (0.1%) was used to determine the maximum amount of liposomal translocation per assay and was set to F₁₀₀ 100%.

### BAX Conformation Change Assay Using Anti-6A7 Immunoprecipitation

Exposure of the 6A7 epitope of BAX was assessed by immunoprecipitation with a 6A7-domain-specific antibody purchased from Santa Cruz (SC-23959). Protein G beads (50 µL, Santa Cruz) were washed three times with 3% BSA in PBS and incubated with 15 µL 6A7 antibody at 4° C. for 1 hr. Recombinant full-length BAX (10 µM) was incubated with 4 equivalents of BIM-BH3 peptide alone and in the presence of 5 or 10 equivalents of EO for 15 min at room temperature. Incubation of full-length recombinant BAX with 0.1% Triton X served as a positive control for exposure of the 6A7 epitope. After incubation, 10 µL of each reaction was transferred to the protein G beads pre-loaded with the anti-6A7 antibody, and 1 µL was reserved as a loading control. After 90 minutes of incubation at 4° C. beads were collected and washed three times with 500 pL of 3% BSA in PBS and solubilized with 25 µL LDS/DTT loading buffer. Samples were resolved by SDS-PAGE electrophoresis and western blot analysis with an anti-BAX yth-2D2 antibody (Invitrogen).

### Western Blotting and Protein Quantification

BAX samples were electrophoretically separated on 4-12% NuPage (Invitrogen) gels, transferred to mobilon-FL PVDF membranes (Millipore) and subjected to immunoblotting. For visualization of proteins with Odyssey Infrared Imaging System (LI-COR Biosciences), membranes were blocked in PBS containing 2.5% milk powder. Primary BAX yth-2d2 antibody (R&D Systems, Cat # 2282-MC-100) was incubated overnight at 4° C. in a 1:1,000 dilution. After washing, membranes were incubated with an IRdye800-conjugated goat anti-mouse IgG secondary antibody (LI-COR Biosciences, Cat # 926-68022) in a 1:5,000 dilution. Protein was detected with the Odyssey Infrared Imaging System. Densitometry of protein bands was acquired using a LI-COR Odyssey scanner. Quantification and analysis were performed using the Western Analysis tool from the Image Studio 3.1 software.

### NMR Samples and Spectroscopy

The uniformly ¹⁵N-labeled protein samples were prepared by growing the bacteria in a minimal medium as previously described (Uchime, O. et al. J Biol Chem 291, 89-102, (2016)). Unlabeled and ¹⁵N-labeled protein samples were prepared in 50 mM potassium phosphate, 50 mM NaCl solution at pH 6.0 in 10% D₂O. All experiments were performed using an independent sample for each experimental measurement as a 400 µL sample in a 5-mm Shigemi; all samples were DMSO matched with 2% d₆-DMSO. Correlation 1H-15N-HSQC spectra were recorded on ¹⁵N-labeled BAX at 50 pM in the presence and absence of 100 pM of EO. NMR spectra were acquired at 25° C. on a Bruker 600 MHz spectrometer equipped with a cryoprobe, processed using TopSpin, and analyzed using NMRView. BAX cross-peak assignments were applied as previously reported (Gavathiotis, E., et al. Mol Cell 40, 481-492 (2010)). The weighted average chemical shift perturbation (CSP) was calculated as √(Δδ¹H)²+( Δδ¹⁵N/5)²)/2 in p.p.m. The absence of a bar indicates no chemical shift difference, the presence of a proline, or a residue that is overlapped or missing and, therefore, not used in the analysis. The significance threshold for backbone amide chemical shift changes was calculated based on the average chemical shift across all residues plus 0.5 or 1 s.d. (Marintchev, A., et al. Methods Enzymol. 430, 283-331). Solvent-accessible surface area was probed by the addition of 10 mM by-TEMPO (Sigma) to 50 pM ¹⁵N-labeled BAX with and without 100 pM EO measured using standard ¹H-¹⁵N-HSQC with an increased recycle delay of 2 S (Garner, T.P., et al. Nat Chem Biol 15, 322-330 (2019); Morin, A. et al. eLife 2, e01456 (2013)). PRE was calculated as the ratio of peak intensities of BAX in the presence of by-TEMPO to BAX without by-TEMPO (% intensity). Mapping of chemical shifts and PRE data onto the BAX structure was performed with PyMOL (Schrödinger, LLC, 2018-2019). Software was made available through the SBGrid collaborative network (Morin, A. et al. eLife 2, e01456 (2013)).

### NMR-Based Docking Calculations and Molecular Dynamics

NMR-guided docking of EO into the NMR structure of BAX (PDB ID: 1F16) was performed using induced-fit docking (IFD, Schrödinger LLC, 2018) with extra precision (XP), and a binding site at the midpoint of residues K21, R134, and R145. EO was converted to 3D all-atom structure using LIGPREP (Schrödinger LLC, 2018) and assigned partial charges with EPIK (Schrödinger LLC, 2018). Poses generated were consistent with NMR data and indicated a strong favoring of an ionic interaction between the carboxylate of EO and a basic residue of BAX Mutagenesis was used to elucidate the true pose of EO on the trigger site of BAX. The pose consistent with mutant BAX liposomal release data was most consistent with NMR CSP data. This pose was subjected to 3 independent 100 nsec molecular dynamics (MD) simulations using DESMOND (DESMOND, version 3, Schrödinger LLC, 2017). Three independent 100 ns MD simulations were also performed with the lowest energy BAX structure from the NMR ensemble (PDB ID: 1F16). MD runs were performed in a truncated octahedron SPC water box using OPLS_2005 force field, 300 K, and a constant pressure of 1.0325 bar. Analysis of the trajectory was performed with MAESTRO simulation event analysis tools (Schrödinger LLC, 2018). PyMOL (Schrödinger LLC, 2018-2019) was used for preparing the highlighted poses. The %ΔRMSF for each residue was calculated as %ΔRMSF = ((RMSFE_{EO} -RMSF_{Apo})100/RMSF_{Apo}), where RMSFE0 was the RMSF of an individual MD simulation of EO docked into BAX and RMSFApo is the average RMSF of the apo BAX simulation. Distance frequency histograms were prepared using GraphPad Prism frequency distribution analysis.

### Structural Analysis

Structural analysis was performed in PyMOL (Schrödinger LLC, 2018-2019) and Maestro tools (Schrödinger LLC, 2018-2019).

### Cytochrome C Release Assay

BAX⁻/⁻ or BAK⁻/⁻ mouse embryonic fibroblasts were maintained in DMEM (Life Technologies) supplemented with 10% FBS, 100 U/mL penicillin/streptomycin, 2 mM I-glutamine, and 0.1 mM MEM nonessential amino acids. MEFs (5×10⁴ cells/well) were seeded in a 96 well clear u-bottom plate for 18-24 hr. Media was removed and replaced with media lacking FBS, and cells were treated with varying doses of EO for 2 hours at 37° C. After incubation, the media was removed and replaced with 100 µL reaction buffer modified from MEB buffer (150 mM mannitol, 10 mM HEPES-KOH pH 7.5, 50 mM KCI, 0.02 mM EGTA, 0.02 mM EDTA, 0.1% BSA, 5 mM succinate, 20 µg/mL oligomycin, 10 mM DTT, and 0.00125% digitonin) with and without 5 pM BIM-BH3 peptide and incubated at 30° C. for 45 min. After incubation, an additional 100 µL of reaction buffer was added, and the plate was gently tapped to mix. Cytochrome c release was determined by decanting 50 µL of the supernatant and analyzing with the rat/mouse cytochrome c quantikine ELISA kit (R&D Systems, MCTO) according to the recommended protocol. Percentage inhibition was normalized to BIM-BH3 peptide alone (0%) and untreated cells (100%).

### Cell Viability and Caspase-317 Activation Assays

3T3 cells were maintained in media identical to that of MEFs. 3T3 cells were seeded (1×10⁴ cells/well) in 96-well opaque plates for 18-24 hr. The media was removed and replaced with media lacking FBS, and cells were treated with EO as a 10X stock in H₂O at the indicated doses for 2 hr before addition of 10% FBS. Cells were then treated with 1 µM each of ABT-263 and S63845. Caspase 3/7 activation was measured at 4 hr by addition of the Caspase-Glo 3/7 chemiluminescence reagent in accordance with the manufacturer’s protocol (Promega). Luminescence was detected by an F200 PRO microplate reader (TECAN). Percentage caspase activation was normalized to ABT-263 + S63845 alone (100%) and untreated cells (0%). Viability assays were performed at 24 hr by addition of CellTiter-Glo according to the manufacturer’s protocol (Promega). Luminescence was detected by an F200 PRO microplate reader (TECAN). Percentage viability was normalized to untreated cells (100%).

### Cellular Thermal Shift Assays (CETSA)

BAK KO MEFs were seeded in a 10 cm dish for 18-24 hours or until approximately 80% confluent. The media was removed and replaced with media lacking FBS, and cells were treated with 10 pM EO as a 10X stock in H2O or vehicle for 2 hours. The media was then removed, and cells were harvested using a cell scraper and washed twice with PBS. Cells were then resuspended in PBS to 6×10⁶ cells/mL, and 50 pL was transferred to PCR tubes. Cells were then heated in a Biorad C1000 Touch Thermal Cycler for 3 minutes using a temperature gradient (50, 52.1, 55.4, 59.4, 64.9, 69.2, 72.1, and 74° C.). Cells remaining at room temperature (25° C.) served as a control. All cells were lysed by three cycles of freeze-thawing using liquid nitrogen. Samples were then centrifuged at 2×10⁴ g for 15 minutes. The supernatants were collected and resolved by SDS-PAGE with an N-terminal BAX antibody (Cell Signaling, 2772S). Samples were analyzed and quantified using a Li-Cor Odyssey Clx and normalized to 25° C. (100%) and 74° C.(0%).

### Calculation of Recombinant BAX TM

Purified recombinant BAX (25 pM) was combined with DMSO or EO (1:10 BAX:EO) and loaded into Tycho NT.6 (NanoTemper Technologies) capillaries. First derivative (330/350 nm) melting point curves were generated automatically using the Tycho NT.6 (NanoTemper Technologies). Data was exported and to GraphPad PRISM software for analysis and visualization.

### In Vivo Mouse Platelet Experiments

Wild type black mice strain C57BL/6J were treated with vehicle, ABT-263 (25 mg/kg, single dose), EO (100 mg/kg, then 3 hrs later 50 mg/kg), or a combination of ABT-263 and EO. EO was formulated in dH2O, and ABT-263 was formulated in 10% ethanol, 30% polyethylene glycol 400, and 60% Phosal 50 PG. Both ABT-263 and EO were administered by oral gavage. Blood samples were collected by submandibular bleed before treatment and 24 hours following treatment, and CBC was acquired. Percentage platelet protection was calculated as follows: % Platelet Protection = (100)(Px - mP_{ABT})/(mPv - mP_{ABT}) where Px is the platelet count for a given mouse, mPv is the mean vehicle treated platelet count, and mPABT is the mean ABT-263 treated platelet count. Statistical analysis was performed using one-way ANOVA with select multiple comparisons as shown.

### Statistical Analysis

Statistical significance for pair-wise comparison of groups was determined by 2-tailed Student’s t test unless otherwise noted using GraphPad PRISM software (Graph Pad Inc., CA). P values of less than 0.05 were considered significant.

### Example 2

### Eltrombopag Binds and Inhibits BAX

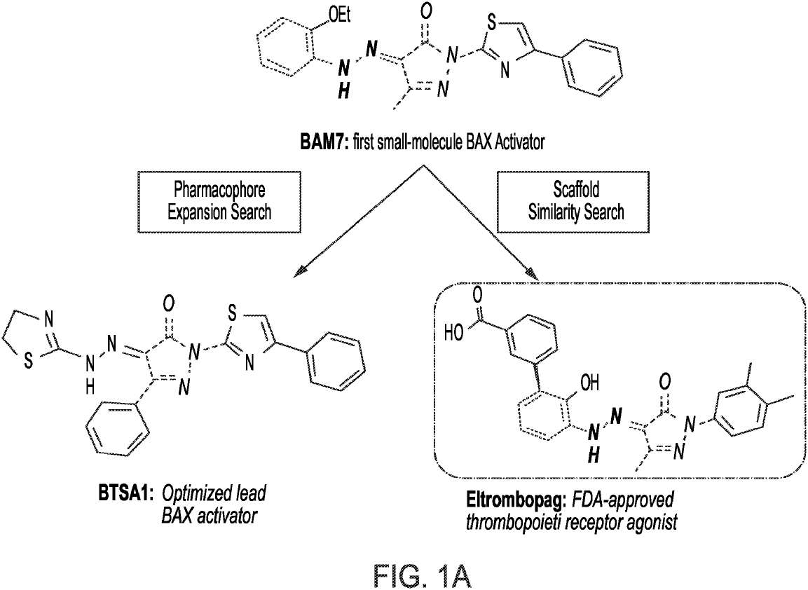

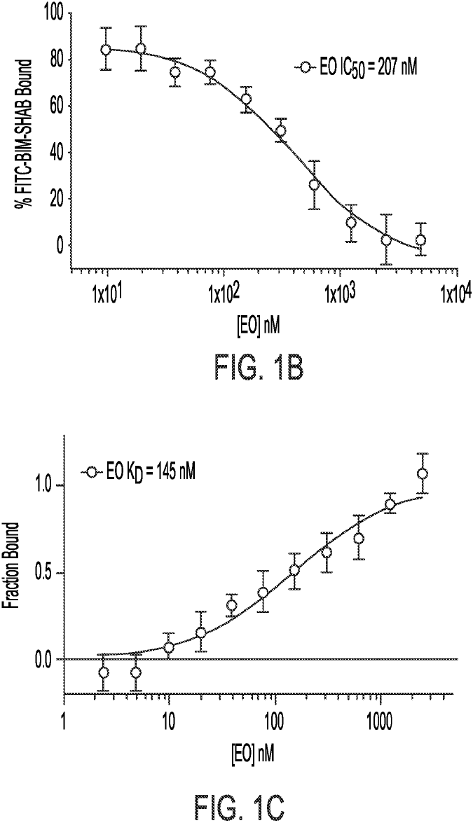

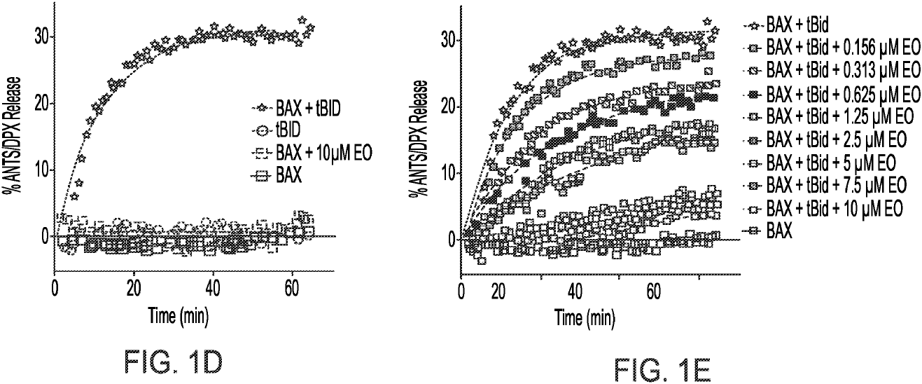

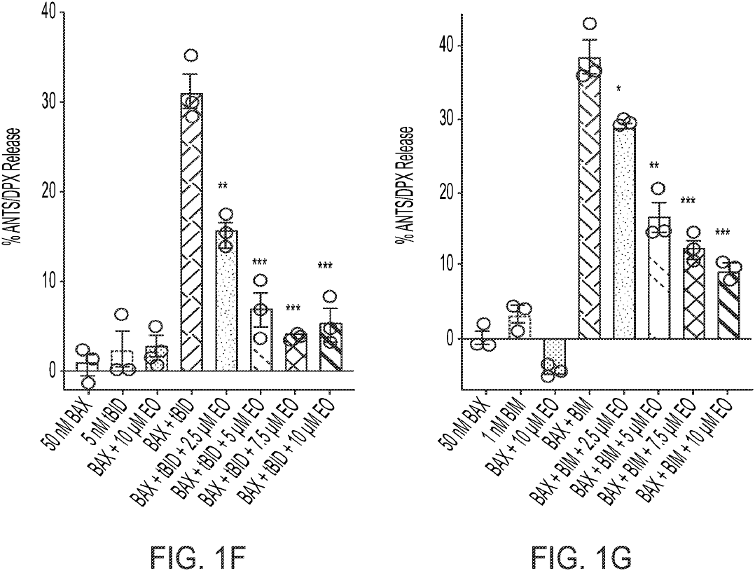

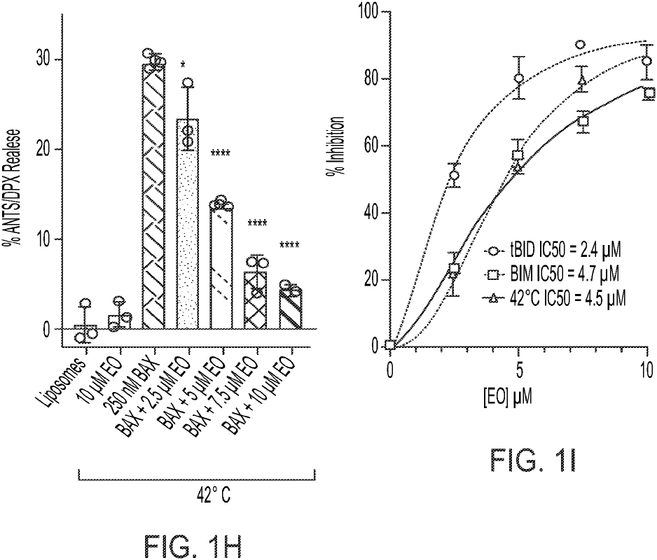

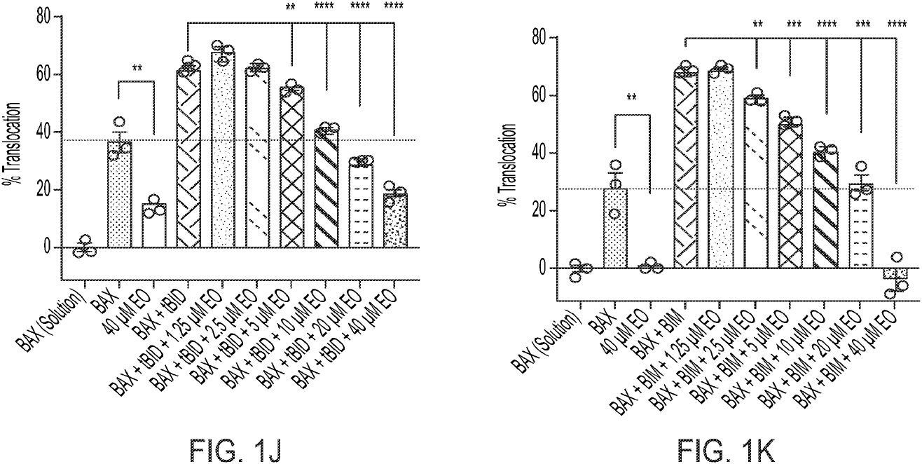

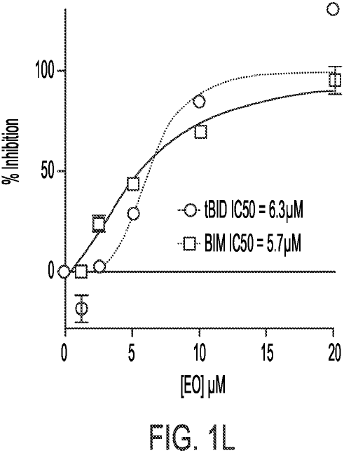

To identify small-molecule BAX modulators, pharmacophore enhancement and substructure similarity computational methods based on the small molecule BAX activator, BAM7 (Gavathiotis, E., et al. Nat Chem Biol 8, 639-645 (2012)), were explored. Specifically, searching a library of FDA-approved small molecules using the 3-methyl pyrazolone and phenylhydrazone core as a query, eltrombopag (EO) was identified as a hit (FIG. 1A). EO bears a striking similarity to BAM7, sharing the 3-methyl pyrazolone and phenylhydrazone core. However, substitutions at either side to this core with dimethylphenyl and benzoic acid markedly distinguish EO from BAM7 and the optimized lead BAX activator, BTSA1 (Gavathiotis, E., et al. Nat Chem Biol 8, 639-645 (2012); Reyna, D.E., et al. Cancer Cell 32 490-505 (2017)) (FIG. 1A).

It was hypothesized that EO would exhibit some binding interaction with the N-terminal BAX trigger site. To test this hypothesis, a competitive fluorescence polarization assay (FPA) (Gavathiotis, E., et al. Nat Chem Biol 8, 639-645 (2012)) based on the interaction between BAX and fluorescently-labeled stapled BIM-BH3 peptide (FITC-BIM-SAHB) was used (Gavathiotis, E., et al. Nature 455, 1076-1081 (2008)). The results show that EO dose-dependently competed FITC-BIM-SAHB with a remarkable half-maximal inhibition IC50 of 207 nM (FIG. 1B). Titrations of BAX to a constant concentration FITC-BIM-SAHB exhibited a decreased affinity in the presence of constant concentrations of EO. Direct binding of EO to BAX was demonstrated using microscale thermophoresis (MST) with a calculated dissociation constant K_{D} of 143 nM (FIG. 1C). The IC50 and K_{D} are consistent with a competitive binding mechanism of EO displacing FITC-BIM-SAHB from the N-terminal trigger site of BAX.

Next, EO’s capacity to modulate BAX activity was evaluated using liposomal release assays. As shown in FIG. 1D, EO exhibited no ability to activate BAX in concentrations, even up to 10 µM. Instead, EO was able to inhibit both tBID- and BIM BH3-mediated BAX activation (FIGS. 1E, 1F, and 1G). Inhibition of tBID-mediated BAX activation by EO was inversely proportional to the concentration of the tBID activator, consistent with competitive inhibition of BH3-activator binding. Furthermore, EO was capable of inhibiting heat-induced BAX activation, indicating that EO can stabilize inactive BAX in addition to blocking of the BAX activation site from BH3-mediated activators (FIG. 1H). EO exhibited similar low micromolar potency in inhibiting BAX activity in liposomal release assay against all stimuli (tBID IC50 = 2.4 µM, BIM IC50 = 4.7 µM, heat IC50 = 4.5 µM) (FIG. 1I).

Prior to permeabilizing membranes, activated BAX must first translocate to the membrane (Lovell, J. F. et al. Cell 135, 1074-1084, (2008)). To explore this earlier step in BAX activation, an NBD-fluorescence-based translocation assay was used. As shown in FIGS. 1J and 1K, EO was capable of inhibiting tBID- and BIM BH3-induced BAX translocation dose-responsively with comparable potency to that of inhibiting liposomal release (tBID IC50 = 6.3 µM, BIM IC50 = 5.7 µM) (FIG. 1L). Similarly, EO was capable of inhibiting heat-induced auto-translocation of BAX, indicating that EO binding stabilizes an inactive conformation of BAX. One of the earliest conformational changes of BH3-mediated BAX activation is the exposure of an N-terminal epitope, requiring opening of the α1-α2 loop from its inactive conformation, which is recognized by an anti-6A7 epitope-specific antibody (Gavathiotis, E., et al. Mol Cell 40, 481-492 (2010); Kim, H. et al. Mol Cell 36, 487-499, (2009)). In an immunoprecipitation assay using the anti-6A7 epitope-specific antibody, BIM-BH3 induced 6A7 exposure as previously shown, but it was inhibited by the presence of EO.

### Example 3

### Eltrombopag Forms Unique Contacts at the BAX Trigger Site

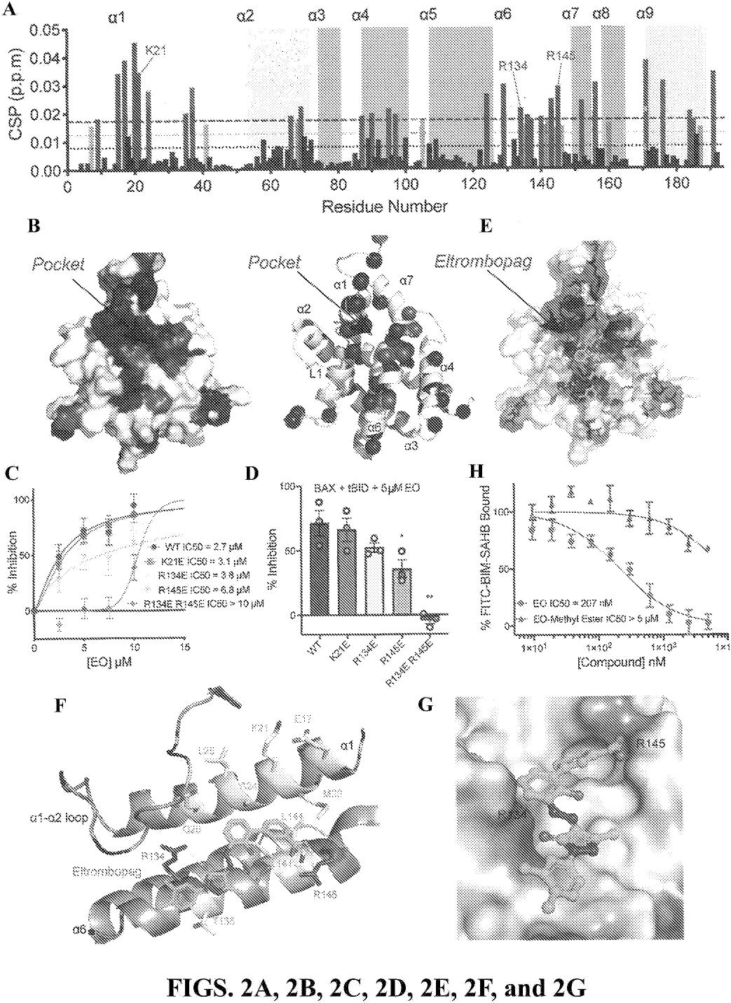

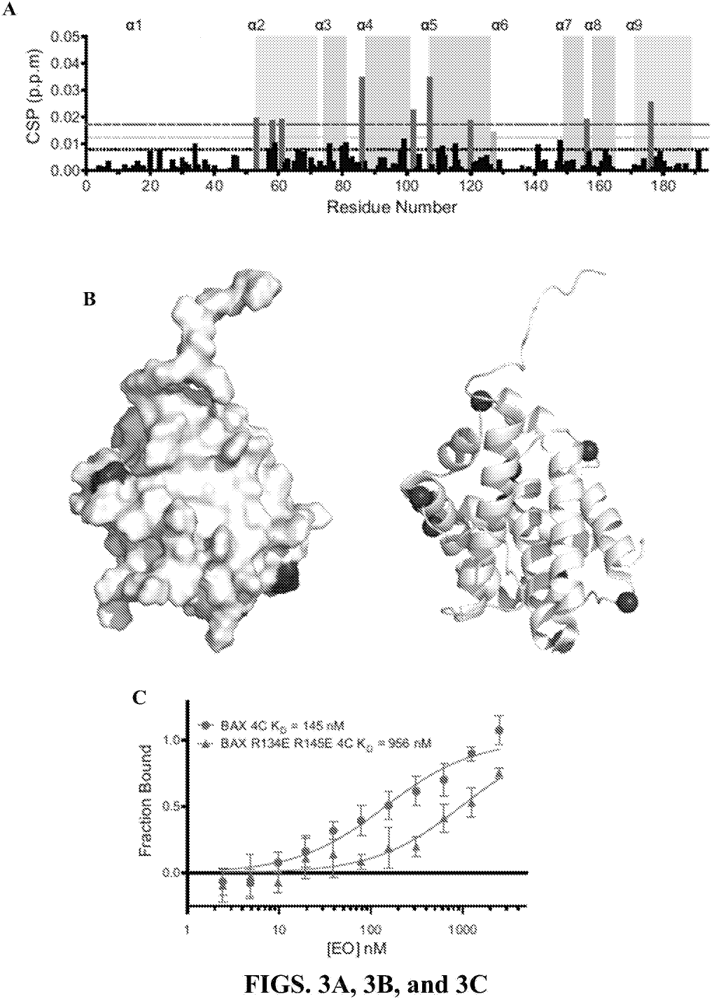

To determine the binding site of EO, 2D ¹H-¹⁵N heteronuclear single quantum coherence (HSQC) NMR was performed. Chemical shift perturbations (CSPs) analysis of ¹⁵N-labeled BAX in the presence of EO indicated small and specific shifts, as with previous NMR studies of BAX (Gavathiotis, E., et al. Nature 455, 1076-1081 (2008); Gavathiotis, E., et al. Mol Cell 40, 481-492 (2010); Gavathiotis, E., et al. Nat Chem Biol 8, 639-645 (2012); Reyna, D.E., et al. Cancer Cell 32 490-505 (2017); Ma, J. et al. Proc Natl Acad Sci USA 109, 20901-20906 (2012); Garner, T.P., et al. Nat Chem Biol 15, 322-330 (2019)). Significant CSPs were localized predominantly to the N-terminal BAX trigger site, specifically the region of α1 and the length of α6 (FIG. 2A). CSPs coalesced to form a contiguous surface with a shallow hydrophobic pocket between α1 and α6 (FIG. 2B). Additional CSPs corresponding to residues in adjacent helices to the trigger site, α4, and α7 but also distant at the C-terminal α9 were observed (FIGS. 2A and 2B). Previous crystal structures of the inactive BAX mutants P168G and W139A suggested that binding at the N-terminal trigger site may modulate BAX activity via local conformational changes at α4, α7, and α9 (Dengler, M.A., et al. Cell Rep. 27 359-373 (2019)). NMR analysis of BAX activation with BIM SAHB and small-molecule trigger site activators also highlighted allosteric sensing in α4, α7, and α9 (Gavathiotis, E., et al. Mol Cell 40, 481-492 (2010); Gavathiotis, E., et al. Nat Chem Biol 8, 639-645 (2012)). Notably, few chemical shift perturbations in N-terminal α1-α2 loop residues were observed, suggesting that its structure remains largely unchanged upon EO binding (FIGS. 2A and 2B). This is in direct contrast with BIM-SAHB and BTSA1 binding, which induce significant CSPs in α1-α2 loop residues and corresponding with a displacement of the α1-α2 loop from the trigger site, a critical step in the activation of BAX (Gavathiotis, E., et al. Mol Cell 40, 481-492 (2010); Gavathiotis, E., et al. Nat Chem Biol 8, 639-645 (2012); Reyna, D.E., et al. Cancer Cell 32 490-505 (2017)).

Next, molecular docking of EO to the BAX surface guided by the CSPs was performed. Ligand preparation for docking of EO using Schrödinger LigPrep at pH 7±1 expectedly yielded EO exclusively with a deprotonated anionic carboxylate group. Docking of EO was performed to a site centered about the three basic residues K21, R134, and R145 and a largely extended surface of BAX at the trigger site to account for potential ambiguity with the NMR data and associated local conformational changes (FIGS. 2A-C and 3A-C). Docking yielded poses which featured ionic interactions between the EO-carboxylate and K21, R134, or R145 as expected.

The EO poses were evaluated by comparing BAX trigger site mutants that would eliminate one of the three basic trigger site residues, K21E, R134E, or R145E, to wild-type (WT). In liposomal release assays, all of the BAX mutants were functional, although R134E and R145E exhibited lower ANTS/DPX release in response to tBID activation. Of the mutants tested, only BAX R145E exhibited a reduced inhibition in response to EO, with an IC50 more than double that of BAX WT (FIGS. 2C-D and 3A-C). Notably, BAX K21E exhibited reduced activation in response to BIM BH3, BAM7, and BTSA1 activators but not reduced inhibition in response to EO (Gavathiotis, E., et al. Nature 455, 1076-1081 (2008); Gavathiotis, E., et al. Mol Cell 40, 481-492 (2010); Gavathiotis, E., et al. Nat Chem Biol 8, 639-645 (2012); Reyna, D.E., et al. Cancer Cell 32 490-505 (2017)).

The loss of EO-mediated BAX inhibition with the R145E mutant suggests that EO forms a critical interaction via the anionic carboxylate with BAX R145. With this known, the EO docking poses were reevaluated, and the top pose featuring an ionic interaction between the EO-carboxylate and sidechain of R145 was analyzed (FIGS. 2E, 2F, and 2G). In addition to the ionic interaction, this pose also features hydrophobic interactions between the biphenyl moiety of EO and the hydrophobic pocket formed by residues L24, M137, G138, and L141 between α1 and α6. Furthermore, the docking pose features contacts at the N-terminal of α6 unique to poses possessing an ionic interaction with R145. Of particular note is a hydrogen bond between R134 sidechain and the carbonyl of the pyrazolone core of EO, which could potentially explain the slight trend towards weaker inhibition of BAX R134E (FIGS. 2C and 2D). Therefore, it was hypothesized that the double mutant BAX R134E R145E would markedly reduce EO inhibition of BAX. Indeed, EO exhibited clearly weak inhibition of BAX R134E R145E with an IC50 >10 µM (FIGS. 2C and 2D). Consistently, in the presence of EO, this double mutant also exhibited minimal CSPs of ¹⁵N-labeled BAX by HSQC-NMR studies and markedly reduced affinity as determined by MST.

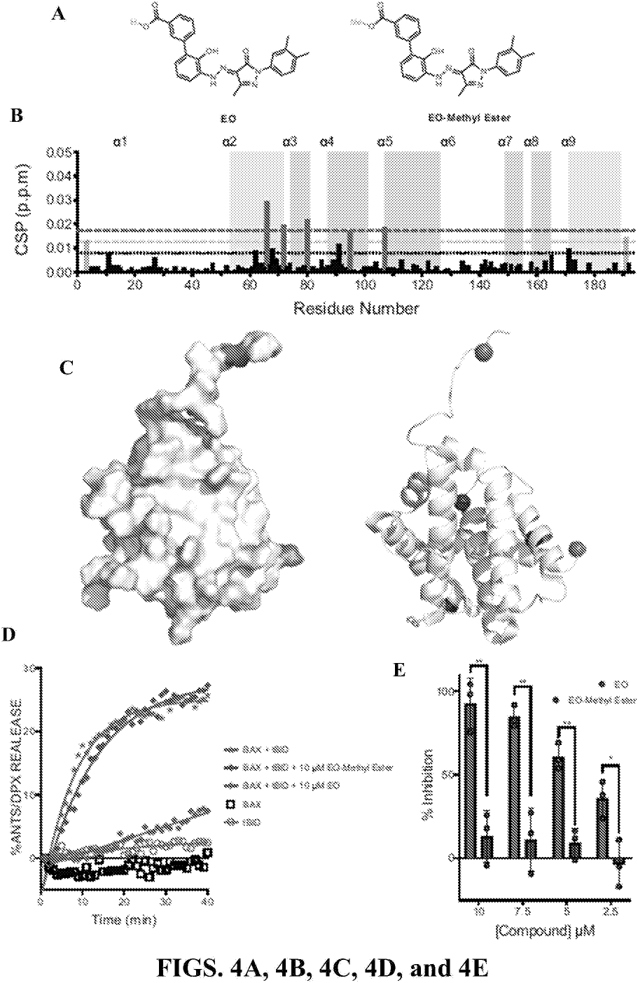

To further probe the specificity of EO for the BAX trigger site and the critical R145 ionic interaction, an EO analog featuring a methyl ester (EO-Methyl Ester) in place of the carboxylic acid was utilized. The addition of a methyl group eliminates the anionic carboxylate as well as adds steric bulk at the site of the critical R145 interaction (FIGS. 2E-2G and 4A-E). EO-Methyl Ester exhibited minimal competition of FITC-BIM-SAHB by FPA (IC50 > 5 µM), indicating a dramatically diminished binding affinity (FIG. 2H). Consistently, EO-Methyl Ester induced minimal CSPs of ¹⁵N-labeled BAX by HSQC-NMR studies and exhibited significantly diminished inhibition of BAX in liposomal release assays.

### Example 4

### Stabilization of Inactive BAX by Eltrombopag

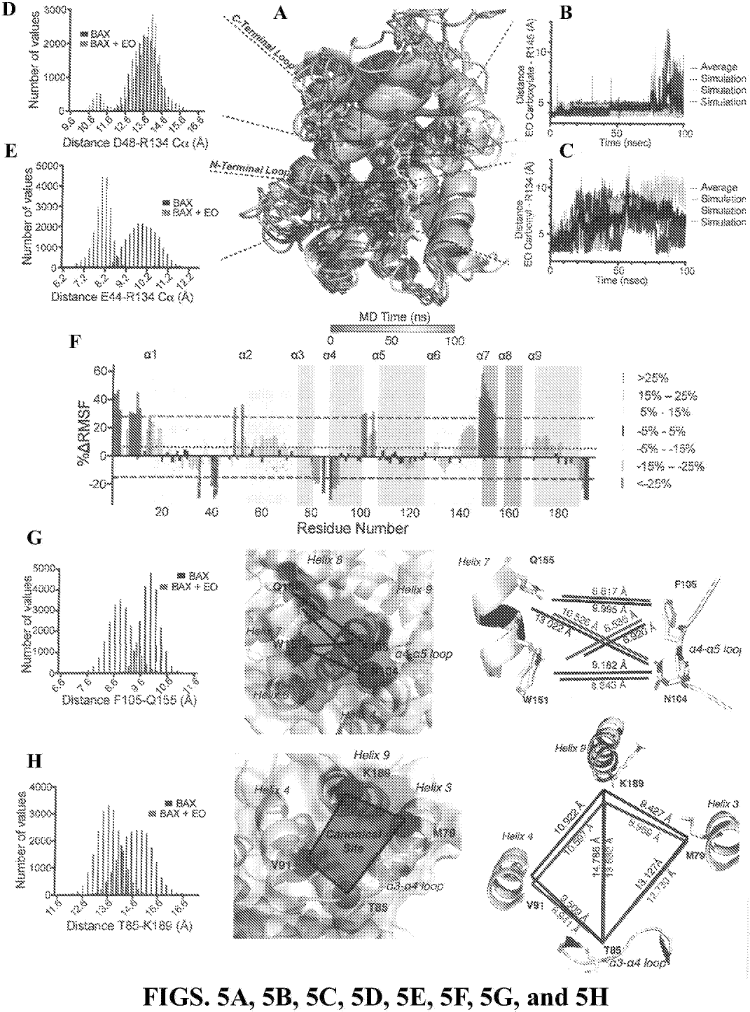

To explore how EO can accomplish inhibition of BAX conformation and activity, three independent molecular dynamics (MD) simulations of the BAX-EO complex and of the inactive BAX structure were performed. The overall structure of BAX was maintained in all six simulations with an average r.m.s. deviation and radius of gyration of 4.17 ± 0.44 Å and 16.36 ± 0.11 Å for the backbone atoms without EO compared to 4.53 ± 0.76 Å and 16.22 ± 0.08 Å bound to EO. In the three MD simulations of the BAX-EO complex, EO remained in a stable conformation (FIG. 5A). The distance between R145 and the EO-carboxylate remained stable throughout the simulation (FIG. 5B). The interaction between R134 and the EO carbonyl was noticeably more dynamic. However, the two groups remained in close proximity throughout the simulation (FIG. 5C).

HSQC CSPs suggested that EO does not cause significant conformational changes to α1-α2 loop, in contrast with other trigger site binders, BTSA1 and BH3 peptides (Gavathiotis, E., et al. Nature 455, 1076-1081 (2008); Gavathiotis, E., et al. Mol Cell 40, 481-492 (2010); Gavathiotis, E., et al. Nat Chem Biol 8, 639-645 (2012); Edwards, A. L. et al. Chem Biol 20, 888-902, (2013)). In the unbound BAX structure, R134 on α6 sits in close proximity to E44 and D48 on α1-α2 loop (Suzuki, M., et al. Cell 103, 645-654 (2000)). The distance between R134 and D48 is approximately equal for both the BAX and BAX-EO simulations (FIG. 5D). However, R134 and E44 remained in closer proximity in simulations of the BAX-EO complex than in BAX alone (FIG. 5E).

To further explore potential conformational changes associated with EO binding to BAX, the percentage change in root mean square fluctuation (RMSF), a measure of the dynamics of each residue in the BAX structure, was analyzed (FIG. 5F). Residues towards the N- and C-terminus of α1-α2 loop exhibit reduced and increased RMSF, respectively, as expected based on α1-α2 loop distances to R134. Two additional regions exhibited dramatic changes in RMSF. The α4-α5 loop and helix α7 exhibit an increase in RMSF, whereas α3-α4 loop and the C-terminal helix α9 exhibit a decrease in RMSF.

The distances between α4-α5 loop and α7 were measured, and reduced distances between most of the residues in the presence of EC were observed, with the exception of the distance between F105 to W151, which increased (FIG. 5G). Furthermore, changes were observed in the distances between R89 and W139 on α4 and α6, respectively, as well as in the distances between R89 and F93 on α6, both of which showed changes in the inactive BAX crystal structures (Dengler, M.A., et al. Cell Rep. 27 359-373 (2019)).

In order for BAX to translocate to the mitochondria, α9 must partially dissociate from the canonical site of BAX (Gavathiotis, E., et al. Nature 455, 1076-1081 (2008); Gavathiotis, E., et al. Mol Cell 40, 481-492 (2010); Gavathiotis, E., et al. Nat Chem Biol 8, 639-645 (2012); Dengler, M.A., et al. Cell Rep. 27 359-373 (2019)). To evaluate this, the distances between four residues forming the boundaries of the opening to the canonical site were measured (FIG. 5H). All of the distances measured were reduced. By approximating the canonical site opening as two triangles, the approximate canonical site opening area of BAX was calculated as 113 Å², and the BAX-EO complex calculated as 102 Å², a reduction of approximately 9%. Taken together, the MD data indicate that EO binding at the BAX trigger site induces direct and allosteric conformational changes consistent with stabilization of the inactive soluble BAX structure.

To independently assess the results of MD simulations, paramagnetic relaxation enhancement (PRE) effects on ¹⁵N-labeled BAX caused by a soluble paramagnetic probe, hy-TEMPO, in the presence and absence of EO were measured. The hy-TEMPO probe is a small sparsely functionalized molecule that can bind nonspecifically to solvent-exposed surfaces and pockets on the surface of BAX. The presence of EO altered the PRE effects not only by directly blocking by-TEMPO binding to the trigger site but by allosterically altering the surface topology of BAX Mapping these changes to the surface of BAX revealed that EO binding protected the trigger site residues in direct contact with EO based on the docking pose, particularly around the hydrophobic pocket formed between α1 and α6. As expected, PRE effects on α1-α2 loop were unchanged in the presence of EO, consistent with this loop remaining closely associated with the trigger site. Furthermore, a reduction in PRE effects was observed on residues surrounding the interface of α7 and α4-α5 loop as well as internal residues of the canonical site such as α3, α5, and α9. This reduction in PRE effects at the interface of α7 and α4-α5 loop is consistent with the closer association predicted by MD simulations (FIG. 5G). Furthermore, the reduction in PRE effects on internal canonical site residues is consistent with stabilization of α9 binding at the canonical site and a narrowing of the canonical site opening as suggested by MD simulations (FIG. 5H).

### Example 5

### Eltrombopag Inhibits BAX-Mediated Apoptosis

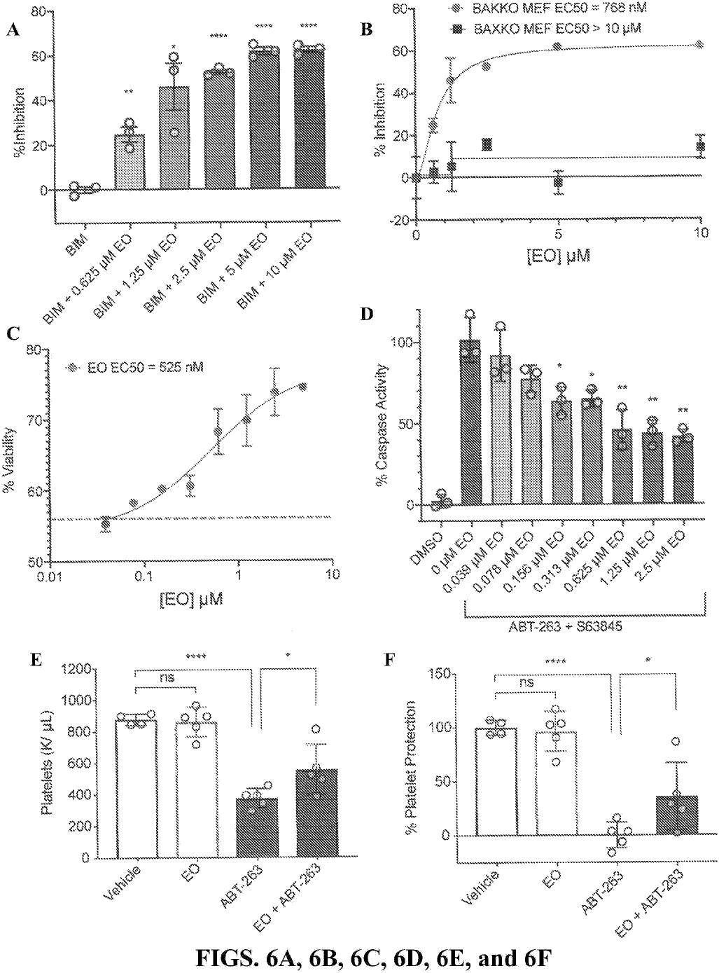

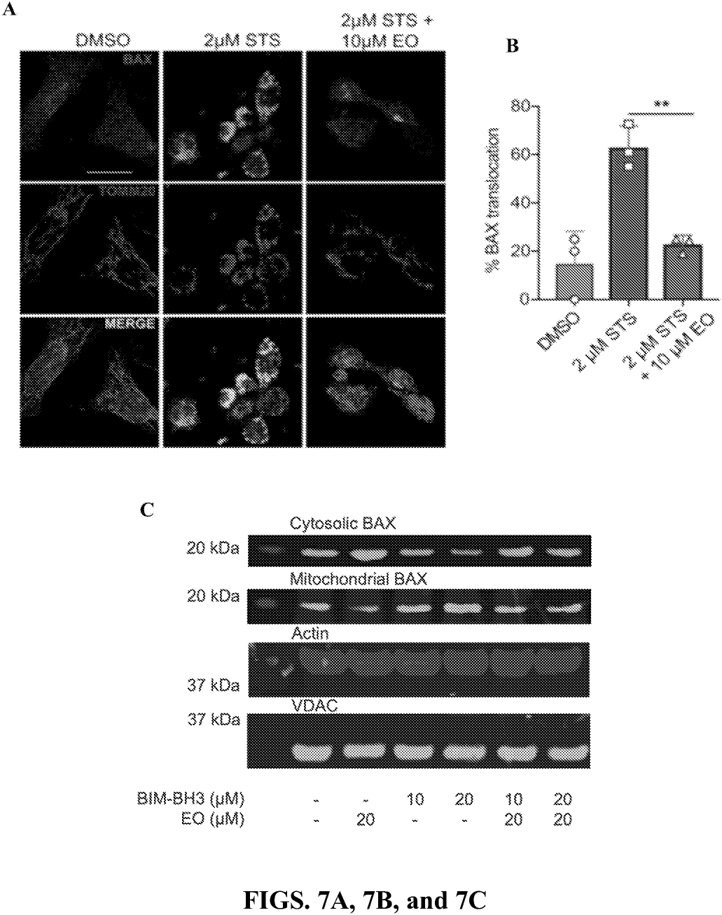

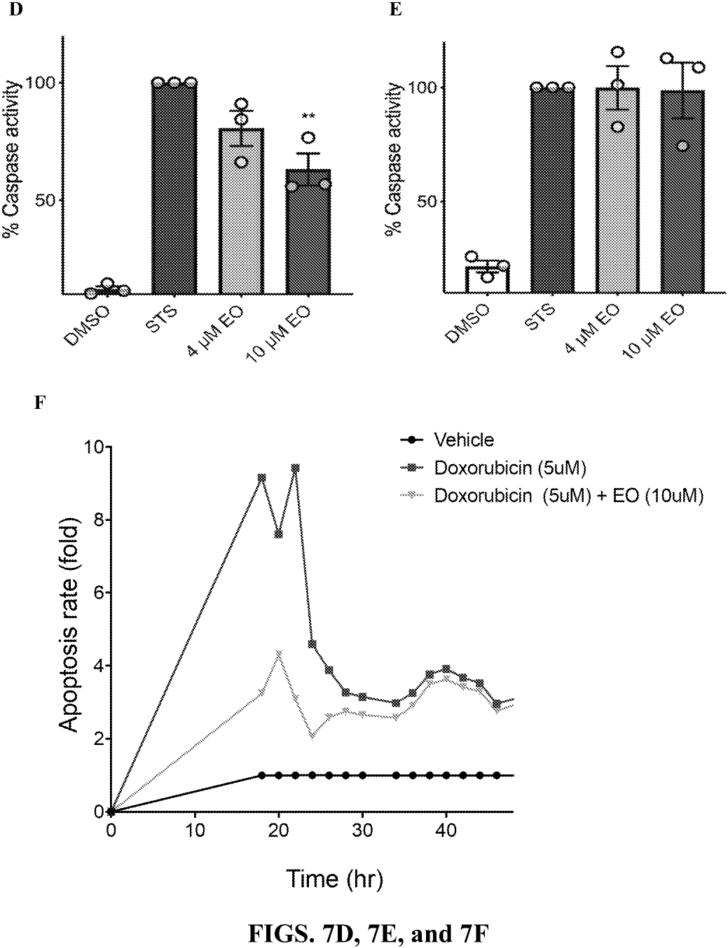

It is important to note that THPO-receptor agonist activity of EO is highly specific to the human and chimpanzee THPO-receptors, making mouse cell lines ideal for studying EO modulation of BAX-dependent activity independent of THPO-mediated effects (Erickson-Miller, C. L. et al. Stem Cells 27, 424-430 (2009)). First, mitochondrial cytochrome c release, a hallmark of BAX activation and BAX-dependent apoptosis, was evaluated. BIM-BH3 induced release of cytochrome c was significantly inhibited by EO in BAK KO (BAK⁻/⁻) MEFs providing direct evidence that EO can inhibit BAX-dependent cytochrome c release (FIG. 6A and FIGS. 7A-7C). EO had no such effect in BAX KO (BAX^{-/-}) MEFs, strongly supporting BAX specificity (FIG. 6B). BAX KO and BAKKO MEFs exhibited similar sensitivity to BIM-BH3 induced cytochrome c release. Next, mitochondrial translocation of cytosolic BAX upon treatment with either BIM BH3 or staurosporine (STS) in BAK KO MEFs was evaluated. It was found that EO is capable of inhibiting BAX translocation (FIGS. 7A-C), consistent with in vitro results (FIGS. 1J-L). Accordingly, EO inhibited STS-induced apoptosis mediated by caspase 3/7 activity in MEFs expressing only BAX, but it had no effect is MEFs expressing only BAK (FIGS. 7D and 7E).

To confirm direct engagement of BAX, Cellular Thermal Shift Assay (CETSA) was performed in BAK KO MEFs. CETSA showed that EO indeed binds BAX in cells by lowering its T_{M} by 9° C. Although decreased TM typically would imply destabilization of BAX by EO, previous studies have demonstrated that inactive BAX mutants can display dramatically reduced T_{M} despite their resistance to activation by BH3-activators, highlighting a critical distinction between the controlled conformational changes of BAX activation and protein unfolding (Dengler, M.A., et al. Cell Rep. 27 359-373 (2019); Robin, A.Y., et al. Structure 26 1346-1359 (2018)). Furthermore, recombinant BAX displayed a comparable reduction in T_{M} in the presence of EO, strongly supporting the CETSA observations.

In addition to inhibition of STS-induced apoptosis, it was additionally found that EO is capable of inhibiting apoptosis (cell death) of human IPSC cardiomyocytes induced by doxorubicin treatment (FIG. 7F), consistent with the protective functional role of BAX inhibition in doxorubicin-induced cardiotoxicity.

Next, whether EO is capable of rescuing cell death in cells expressing both BAX and BAK was determined. 3T3 cells, a murine fibroblast cell line, were treated with a combination of BH3-mimetics navitoclax (ABT-263) and S63845, which only in combination cause significant cytotoxicity in fibroblast cells. Strikingly, cells treated with EO exhibited a dose-dependent rescue of cell viability as well as a corresponding significant reduction of apoptosis mediated by caspase 3/7 activity (FIGS. 6C-6D).

Finally, whether EO can rescue thrombocytopenia induced by the activity of navitoclax was evaluated in mice. The use of navitoclax is limited by on-target toxicity triggering BAK/BAX-mediated platelet apoptosis (Zhang, H. et al. Cell Death Differ 14, 943-951 (2007)). While significant platelet loss was induced by navitoclax within 24 hr, co-administration of navitoclax with EO markedly inhibited platelet loss to acceptable levels (FIGS. 6E and 6F). This EO effect is distinct from its capacity to stimulate platelet production by differentiation of the megakaryocyte precursors and progenitor cells, which requires 5 days to begin (Erickson-Miller, C. L. et al. Stem Cells 27, 424-430 (2009); Jenkins, J. M. et al. Blood 109, 4739-4741 (2007)).

This disclosure showed that EO, an FDA-approved thrombopoietin receptor agonist and iron chelator that is used to increase blood platelet counts in chronic immune thrombocytopenia (Zhang, Y., et al. Clin. Ther. 33 1560-1576 (2011); Vlachodimitropoulou, E. et al. Blood 130, 1923-1933, (2017)), is a direct inhibitor of BAX. This disclosure characterized the mechanism of BAX inhibition and found that EO inhibits BAX-mediated cell death in vitro and in vivo. This unrecognized activity of EO expands its use in biological systems and in diseases of pathological BAX-mediated cell death. EO binds the BAX trigger site distinctly from BAX activators, preventing them from triggering BAX conformational transformation and simultaneously promoting allosteric stabilization of the inactive BAX structure. Accordingly, eltrombopag is capable of inhibiting BAX functional activity and BAX-mediated apoptosis induced by cytotoxic stimuli. The data demonstrated structure-function insights into a mechanism of BAX inhibition and revealed an additional mechanism for eltrombopag that expands its use to diseases of uncontrolled cell death.

What is claimed is:

1. A method of treating or preventing a disorder mediated by BAX in a subject, comprising administering to the subject a therapeutically effective amount of eltrombopag, a pharmaceutically acceptable salt thereof or pharmaceutically acceptable prodrug thereof that binds to a BAX protein and inhibits activation or function of the BAX protein.

2. The method of claim 1, wherein the disorder is associated with increased expression or activation of the BAX protein.

3. The method of claim 1, wherein the disorder comprises a neuronal disorder or an autoimmune disease.

4. The method of claim 3, wherein the neuronal disorder is selected from the group consisting of epilepsy, multiple sclerosis, Alzheimer’s disease, Huntington’s disease, Parkinson’s disease, retinal diseases, macular degeneration, spinal cord injury, Crohn’s disease, head trauma, spinocerebellar ataxias, and dentatorubral-pallidoluysian atrophy.

5. The method of claim 3, wherein the autoimmune disease is selected from the group consisting of multiple sclerosis, amyotrophic lateral sclerosis, retinitis pigmentosa, inflammatory bowel disease (IBD), rheumatoid arthritis, asthma, lupus, septic shock, organ transplant rejection, and AIDS.

6. The method of claim 1, wherein the disorder comprises ischemia, cardiomyopathy, chemotherapy-induced cardiotoxicity, chemotherapy-induced cardiomyopathy, cardiovascular disorders, arteriosclerosis, heart failure, heart transplantation, renal hypoxia, a liver disease, a kidney disease, an intestinal disease, liver ischemia, intestinal ischemia, acute optic nerve damage, glaucoma, chemotherapy-induced ocular toxicity, hepatitis.

7. The method of claim 1, further comprising administering to the subject a second therapeutic agent or therapy.

8. The method of claim 7, wherein the second therapeutic agent is an antiinflammatory agent or an anti-tumor/anti-cancer agent.

9. The method of claim 8, wherein the anti-tumor/anti-cancer agent is navitoclax.

10. The method of claim 7, wherein the second therapeutic agent is administered to the subject before, after, or concurrently with the eltrombopag, a pharmaceutically acceptable salt thereof or pharmaceutically acceptable prodrug thereof.

11. The method of claim 1, wherein the subject was previously administered an anti-cancer therapy.

12. The method of claim 11, wherein the anti-cancer therapy comprises surgery, radiation, chemotherapy, and/or immunotherapy.

13. The method of claim 12, wherein the chemotherapy comprises a therapeutic agent that inhibits Bcl-xL.

14. The method of claim 13, wherein the therapeutic agent that inhibits Bcl-xL is navitoclax.

15. The method of claim 1, wherein the subject is a mammal.

16. The method of claim 1, wherein the subject is a human.

17. The method of claim 1, wherein the eltrombopag, a pharmaceutically acceptable salt thereof or pharmaceutically acceptable prodrug thereof is administered intratumorally, intravenously, subcutaneously, intraosseously, orally, transdermally, in sustained release, in controlled release, in delayed release, as a suppository, or sublingually.

18. The method of claim 1, wherein the eltrombopag, a pharmaceutically acceptable salt thereof or pharmaceutically acceptable prodrug thereof, is administered prophylactically or therapeutically.

19. A method of treating or ameliorating a symptom of thrombocytopenia associated with treatment targeting Bcl-xL, comprising:
(i) selecting a subject having a condition treatable by a therapeutic agent that inhibits Bcl-xL; and
(ii) administering to the subject a therapeutically effective amount of eltrombopag, a pharmaceutically acceptable salt thereof or pharmaceutically acceptable prodrug thereof, in combination with a therapeutically effective amount of the therapeutic agent.

20. The method of claim 19, wherein the therapeutic agent that inhibits Bcl-xL is navitoclax.

21. The method of claim 19, wherein the condition is a cancer.

22. The method of claim 19, wherein the therapeutic agent is administered to the subject before, after, or concurrently with the eltrombopag, a pharmaceutically acceptable salt thereof or pharmaceutically acceptable prodrug thereof.

23. The method of claim 19, wherein the therapeutic agent or the eltrombopag, a pharmaceutically acceptable salt thereof or pharmaceutically acceptable prodrug thereof, is administered in one or more doses to the subject.

24. A method of inhibiting BAX-mediated apoptosis in a cell, comprising administering to the cell expressing a BAX protein an effective amount of eltrombopag, a pharmaceutically acceptable salt thereof or pharmaceutically acceptable prodrug thereof that binds to the BAX protein and inhibits activation or function of the BAX protein.

25. The method of claim 24, wherein the BAX-mediated apoptosis is caused by doxorubicin-induced cardiotoxicity.

26. A method of inhibiting activation or function of a BAX protein in a cell, comprising administering to the cell expressing a BAX protein an effective amount of eltrombopag, a pharmaceutically acceptable salt thereof or pharmaceutically acceptable prodrug thereof that binds to the BAX protein.

27. The method of claim 24, wherein the cell is a neuronal cell or a cardiac cell.

28. The method of claim 24, wherein the activation of BAX protein is mediated by Bim, Bid, Bmf, Puma, or Noxa.


SEQUENCE LISTING (3 sequences)

SEQ ID NO: 1 - protein, 20 aa, Artificial
  Synthetic
  S5 (10)..(11): Hydrocarbon-stapled peptide corresponding to the BH3 domain of BIM, BIM SAHBA2, where S5 represents the non-natural amino acid inserted for olefin metathesis
  S5 (15)..(16): Hydrocarbon-stapled peptide corresponding to the BH3 domain of BIM, BIM SAHBA2, where S5 represents the non-natural amino acid inserted for olefin metathesis
EIWIAQELRSIGDSFNAYYA

SEQ ID NO: 2 - protein, 24 aa, Artificial
  Synthetic
RPEIWIAQELRRIGDEFNAYYARR

SEQ ID NO: 3 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG